(19) United States
(12) Patent Application Publication
(10) Pub. No.: US 2007/0213312 A1
(43) Pub. Date: Sep. 13, 2007

(54) Hif-1 Inhibitors and Methods of Use Thereof

(76) Inventors: Wayne B. Harris, Stone Mountain, GA (US); Jay N. Umbreit, Wilmington, NC (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/578,131

(22) PCT Filed: Nov. 8, 2004
(86) PCT No.: PCT/US04/37090

§ 371 Date: Feb. 14, 2007

Related U.S. Application Data
(60) Provisional application No. 60/518,146, filed on Nov. 7, 2003.

(51) Int. Cl. A61K 31/555; A61K 31/341; C12N 15/01

(52) U.S. Cl. 514/185; 435/441; 514/472

(57) ABSTRACT

Methods of treating a cancer or tumor, chemopreventative methods of prophylactically treating cancers or tumors, pharmaceutical compositions, methods for the treatment or prevention of a hypoxia-related pathology, methods of modulating HIF-1 activity in a cell, methods of downregulating HIF-1 activity in a cell, methods of treating or preventing cancer or a tumor in a host, and methods of modulating gene transcription in a cell are described.

### CROSS-REFERENCE TO RELATED APPLICATION

This application claims priority to copending U.S. provisional application entitled, “A Method For Down-Regulating Hypoxia-Inducible Factor 1 Alpha Under Hypoxic Conditions,” having Ser. No. 60/518,146, filed Nov. 7, 2003, which is entirely incorporated herein by reference.

### TECHNICAL FIELD OF THE INVENTION(S)

The present disclosure is generally related to compositions and agents and methods for administration to hosts and, more particularly, is related to a compositions and agents designed for treatment of conditions and/or diseases related to Hypoxia Inducible Factor-1 (HIF-1).

### BACKGROUND

According to the American Cancer Society, approximately 1.3 million Americans are estimated to be diagnosed with invasive cancer in 2003. The National Cancer Institutes estimates that approximately 8.9 million Americans had a history of cancer in 2003, and approximate 1,500 cancer-related deaths per day are expected in 2003. Because of the staggering number of cancer-related deaths and new cases, new medicines and methods of treatment are needed. Although recent advances have increased our understanding of some of the mechanisms leading to cancer, effective treatments for cancer remain in high demand.

Cancer can be defined as an abnormal growth of tissue characterized by a loss of cellular differentiation. This term encompasses a large group of diseases in which there is an invasive spread of undifferentiated cells from a primary site to other parts of the body where further undifferentiated cellular replication occurs, which eventually interferes with the normal functioning of tissues and organs.

Cancer can be defined by four characteristics which differentiate neoplastic cells from normal ones: (1) clonality—cancer starts from genetic changes in a single cell which multiplies to form a clone of neoplastic cells; (2) autonomy—biochemical and physical factors that normally regulate cell growth, do not do so in the case of neoplastic cells; (3) anaplasia—neoplastic cells lack normal differentiation which occurs in nonmalignant cells of that tissue type; (4) metastasis—neoplastic cells grow in an unregulated fashion and spread to other parts of the body.

Each cancer is characterized by the site, nature, and clinical cause of undifferentiated cellular proliferation. The underlying mechanism for the initiation of cancer is not completely understood; however, about 80% of cancers may be triggered by external stimuli such as exposure to certain chemicals, tobacco smoke, ultra violet rays, ionizing radiation, and viruses. Development of cancer in immunosuppressed individuals indicates that the immune system is an important factor controlling the replication and spread of cancerous cells throughout the body.

The high incidence of cancer in certain families, though, suggests a genetic disposition towards development of cancer. The molecular mechanisms involved in such genetic dispositions fall into a number of classes including those that involve oncogenes and suppressor genes.

Proto-oncogenes are genes that code for growth promoting factors necessary for normal cellular replication. Due to mutation, such proto-oncogenes are inappropriately expressed—and are then termed oncogenes. Oncogenes can be involved in malignant transformation of the cell by stimulating uncontrolled multiplication.

Suppressor genes normally act by controlling cellular proliferation through a number of mechanisms including binding transcription factors important to this process. Mutations or deletions in such genes contribute to malignant transformation of a cell.

Malignant transformation develops and cancer results because cells of a single lineage accumulate defects in certain genes such as proto-oncogenes and suppressor genes responsible for regulating cellular proliferation. A number of such specific mutations and/or deletions must occur in a given cell for initiation of uncontrolled replication. It is believed that genetic predisposition to a certain type of cancer results from inheritance of genes that already have a number of mutations in such key regulatory genes and subsequent exposure to environmental carcinogens causes enough additional key mutations or deletions in these genes in a given cell to result in malignant transformation. Changes in other types of genes could further the ability of tumors to grow, invade local tissue, and establish metastases at distant body sites.

Current treatments of cancer and related diseases have limited effectiveness and numerous serious unintended side effects. Cancer therapy is currently divided into many categories including surgery, radiation therapy, chemotherapy, bone marrow transplantation, stem cell transplantation, hormonal therapy, immunotherapy, antiangiogenic therapy, targeted therapy and gene therapy and others. These treatments have largely progressed incrementally during more than thirty years of intensive research to discover the origins of cancer and devise improved therapies for cancer and related diseases.

Current research strategies emphasize the search for effective therapeutic modes with less risk, including the use of natural products and biological agents. This change in emphasis has been stimulated by the fact that many of the consequences, to both patients and their offspring, of conventional cancer treatment result from their actions on genetic material and mechanisms. Efforts continue to discover both the origins of cancer at the genetic level and correspondingly new treatments, but such interventions also may have serious unanticipated effects.

Hypoxia is a major hindrance to effective solid tumor therapy. The microenvironment of rapidly growing solid tumors is associated with increased energy demand and diminished vascular supply, resulting in focal areas of prominent hypoxia and regions with reduced oxygen tensions (Folkman J., What is the evidence that tumors are angiogenesis dependent? J Natl Cancer Inst 82, 4-6(1989)). Tissue oxygen electrode measurements taken in cancer patients showed a median range of oxygen partial pressure of 10 to 30 mmHg, with a significant proportion of readings below 2.5 mmHg, whereas those in normal tissues ranged from 24 to 66 mg (Vaupel P. W. Oxygenation of solid tumors. In Drug Resistance in Oncology. Teicher, B. A. (ed.) 53-85 (Marcel Dekker, New York, 1993). (Gray L. H. et al. Concentration of oxygen dissolved in tissues at the time of irradiation as a factor in radiotherapy. Br J Radiol 26, 638-648 (1953). Radiotherapy is severely compromised in its ability to kill hypoxic tumor cells because oxygen is the mediator of the therapeutic effect of ionizing radiation.

Hypoxia (and possibly hypoxia-associated deficiencies in other nutrients such as glucose) causes tumor cells to stop or slow their rate of progression through the cell cycle (Amellem O, Pettersen E O. Cell inactivation and cell cycle inhibition as induced by extreme hypoxia: the possible role of cell cycle arrest as a protection against hypoxia-induced lethal damage. Cell Prolif 24, 127-141 (1991)). Because anticancer drugs are generally more effective against rapidly proliferating cells than slowly or non-proliferating cells, this slowing of cell proliferation leads to decreased cell killing. Furthermore, chemotherapeutic drugs that are delivered systemically have a limited capacity for diffusion into the tumor. Hypoxic regions are therefore exposed to less drug than the oxygenated cells proximal to the vessels. Moreover, the multidrug resistance (MDR1) gene product P-glycoprotein is induced by ambient hypoxia (Comerford K. M. et al. Hypoxia-inducible factor-1-dependent regulation of the multidrug resistance (MDR1) gene. Cancer Res 62, 3387-94(2002)). Hypoxia also drives genetic changes in tumors such as loss of p53 tumor suppressor gene (Brown, J. M., and Giaccia, A. J. The unique physiology of solid tumors: opportunities (and problems) for cancer therapy. Cancer Res. 58(7):1408-16 (1998)).

Finally, hypoxic regions are expected to be less amenable to immunotherapy due to distance from nearby vessels and compromised lymphocyte function in a hypoxic environment. Tumor cells in this aberrant environment are therefore often resistant to radio- and chemotherapy (reviewed in Brown, J. M., and Giaccia, A. J. The unique physiology of solid tumors: opportunities (and problems) for cancer therapy. Cancer Res. 58(7):1408-16 (1998).

Accordingly, there is a need for new and effective treatments for cancer. In particular, there is a need for new and effective treatments that address hypoxia and its role in hyperproliferative pathologies.

### SUMMARY

Briefly described, embodiments of the present disclosure include methods of treating cancer or tumor, chemopreventative methods of prophylactically treating cancers or tumors, pharmaceutical compositions, methods for the treatment or prevention of a hypoxia-related pathology, methods of modulating HIF-1 activity in a cell, methods of downregulating HIF-1 activity in a cell, methods of treating or preventing cancer or a tumor in a host, and methods of modulating gene transcription in a cell.

In an embodiment of the method of treating cancer or tumor, among others, includes: administering to a host in need of treatment an effective amount of at least one HIF-1 inhibitor composition, wherein the HIF-1 inhibitor composition is a bidentate zinc chelate.

In an embodiment of the chemopreventative method of prophylactically treating cancers or tumors, among others, includes: administering to a host in need of treatment an effective amount of at least one bidentate zinc chelate (e.g., complexes C1-C10 as shown in the FIGURES).

In an embodiment of the pharmaceutical composition, among others, includes: at least one bidentate zinc chelate in combination with a pharmaceutically acceptable carrier, wherein the at least one bidentate zinc chelate is present in a dosage level effective to treat cancers or tumors (e.g., complexes C1-C10 as shown in the FIGURES).

In an embodiment of the method for treatment or prevention of a hypoxia-related pathology, among others, includes: administering to a host in need of such treatment an HIF-1 inhibiting amount of at least one of the bidentate zinc chelate (e.g., complexes C1-C10 as shown in the FIGURES).

In an embodiment of modulating HIF-1 activity in a cell, among others, includes: contacting the cell with an HIF-1 inhibiting amount of at least one of the bidentate zinc chelate (e.g., complexes C1-C10 as shown in the FIGURES).

In an embodiment of downregulating HIF-1 activity in a cell, among others, includes: contacting the cell with an HIF-1 inhibiting amount of at least one of the bidentate zinc chelate (e.g., complexes C1-C10 as shown in the FIGURES).

In an embodiment of treating or preventing cancer or a tumor in a host, among others, includes: administering to the host a HIF-1 inhibiting amount of at least one of the bidentate zinc chelate (e.g., complexes C1-C10 as shown in the FIGURES).

In an embodiment of modulating gene transcription in a cell, among others, includes: contacting the cell with an HIF-1 inhibiting amount of at least one of the zinc bidentate complex (e.g., complexes C1-C10 as shown in the FIGURES).

Other systems, methods, features, and advantages of the present disclosure will be or will become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an embodiment of a beta-diketone compound.

FIG. 2 illustrates an embodiment of a bidentate zinc chelate.

FIG. 3 illustrates dibenzoylmethane.

FIG. 4 illustrates an embodiment of HIF-1 inhibitor compositions.

FIG. S illustrates an embodiment of HIF-1 inhibitor compositions.

FIG. 6 illustrates an embodiment of HIF-1 inhibitor compositions.

FIG. 7 illustrates an embodiment of HIF-1 inhibitor compositions.

FIG. 8 illustrates an embodiment of HIF-1 inhibitor compositions.

FIG. 9 illustrates an embodiment of HIF-1 inhibitor compositions.

FIG. 10 illustrates an embodiment of HIF-1 inhibitor compositions.

FIG. 11 illustrates an embodiment of HIF-1 inhibitor compositions.

FIG. 12 illustrates an embodiment of HIF-1 inhibitor compositions.

FIG. 13 illustrates embodiments of functional groups Ar1 and Ar2.

FIG. 14 illustrates the role of oxygen in regulating HIF-1.

FIG. 15 illustrates Western blot analysis of HIF-1 in HEK 293 human embryonic kidney cells exposed to DBM, Zn²⁺ and ambient oxygen.

FIG. 16 illustrates Western blot analysis of HIF-1α in (A) HT144 human melanoma cells and (B) HEK 293 cells exposed to DBM and Zn²⁺ under hypoxic conditions.

FIG. 17 illustrates analysis by RT-PCR of HIF-1α mRNA levels in HEK 293 cells exposed to DBM and Zn²⁺ on under normoxic conditions.

FIG. 18 illustrates Western blot analysis of HIF-1α in HEK 293 cells exposed to DBM, Zn²⁺ and MG-132 (a proteosome inhibitor) under normoxic conditions.

FIG. 19 illustrates Western blot analysis of HIF-1α in VHL (−/−) RCC-4 human renal cell carcinoma cells exposed to DBM, Zn²⁺ and MG-132 under normoxic conditions.

FIG. 20 illustrates comparisons of the prolyl-4-hydroxylation reaction catalyzed by HIF P4H (A) and the proteolytic reaction catalyzed by Zn²⁺ metalloproteases (B). The --Proline-- and --Pro4OH-- residues in (A) are amino acids in HIF-1α, while (B) represents a buffer with a free pair of electrons (R32 and R33 are amino acids).

FIG. 21 illustrates (A) DBM (diketo form) (B) DBM (enol form) (C) 2-Oxoglutarate (2-OG) (D) the active site of calcineurin A, (E) the proposed active site for the “HIF Protease”, and (F) the active site of native HIF P4H.

### DETAILED DESCRIPTION

The present disclosure may be understood more readily by reference to the following detailed description and the Examples included therein.

Before the present compounds, compositions, and methods are disclosed and described, it is to be understood that this disclosure is not limited to specific pharmaceutical carriers, or to particular pharmaceutical formulations or administration regimens, as such may, of course, vary. It is also to be understood that the terminology used herein is for the purpose of describing particular embodiments only and is not intended to be limiting.

Definitions

As used herein, the term “host” or “organism” includes both humans, mammals (e.g., cats, dogs, horses, etc.), and other living species that are in need of cancer and/or cancer related treatments. A living organism can be as simple as, for example, a single eukaryotic cell or as complex as a mammal. Hosts that are “predisposed” to cancer and cancer-related conditions can be defined as hosts that do not exhibit overt symptoms of one or more of these conditions but that are genetically, physiologically, or otherwise at risk of developing one or more of these conditions. Thus, compositions and effector agents of the present disclosure can be used prophylactically as chemopreventative agents for these conditions. Further, a “composition” or “agent” can include one or more chemical compounds and/or agents, as described below.

As used herein, “host cells” include non-cancerous and cancerous cells. “Cancerous cells” include, but are not limited to, cancer cells, neoplastic cells, neoplasia, tumors, and tumor cells, which exhibit relatively autonomous growth, so that they exhibit an aberrant growth phenotype, characterized by a significant loss of control of cell proliferation.

The term “HIF-1 inhibitor” means a compound, pharmaceutically acceptable salt, prodrug, or derivative thereof that inhibits the biological activity of HIF-1, interferes with the HIF-1 signal transduction pathway, and/or down regulates expression and/or availability of HIF-1 in a cell or organism.

The term “hypoxia-related pathology” means a pathology that is caused, at least in part, either directly or indirectly, by conditions of below typical physiological amounts of oxygen. The term includes cancer, cancer metastasis, ischemia, stroke and related conditions, diseases, or syndromes.

“Down regulation” or “down regulating” can be defined as a decrease in the number of ligand receptors or other cellular proteins within or on the surface of a host cell. Down regulation occurs after host cells have been exposed to an effector agent, either as a result of a direct interaction of the effector agent with the receptor or other protein, or through indirect interactions.

Down regulation of cellular proteins may be induced by any cellular perturbation that results in a decrease in protein production or synthesis or an increase in protein degradation. Cellular protein synthesis occurs through the sequential steps of transcription and translation. Transcription is defined as the synthesis of ribonucleic acid (RNA) from a deoxyribonucleic acid (DNA) template. Translation is defined as the synthesis of a protein directed by messenger RNA (mRNA). In general, lysosomes and ubiquitin are the two major pathways for the degradation of cellular proteins. Proteins that undergo ubiquitination such as hypoxia inducible factor-1α are degraded by a subcellular protein complex known as the proteosome.

The term “derivative” means a modification to the disclosed compounds including, but not limited to, hydrolysis, reduction, or oxidation products, of the disclosed compounds. Hydrolysis, reduction, and oxidation reactions are known in the art.

The term “therapeutically effective amount” as used herein refers to that amount of the compound being administered which will relieve to some extent one or more of the symptoms of the disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing and/or reducing the metastasis of cancer cells, (4) relieving to some extent (or, preferably, eliminating) one or more symptoms associated with a pathology related to or caused in part by unregulated or aberrant cellular division, including for example, cancer, (5) prevention the formation of cancer by application of the compound (like sun screen to protect against cancer), and/or (6) to prevent the chain of events downstream of an initial ischemic condition which leads to the pathology.

“Pharmaceutically acceptable salt” refers to those salts that retain the biological effectiveness and properties of the free bases and which are obtained by reaction with inorganic or organic acids such as, but not limited to, hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples of excipients include, but are not limited to, calcium carbonate, calcium phosphate, various sugars and types of starch, cellulose derivatives, gelatin, vegetable oils and polyethylene glycols.

As used herein, “treat”, “treating”, and “treatment” are an approach for obtaining beneficial or desired clinical results. For purposes of embodiments of this disclosure, beneficial or desired clinical results include, but are not limited to, preventing the disease from occurring in an animal that may be predisposed to the disease but does not yet experience or exhibit symptoms of the disease (prophylactic treatment), alleviation of symptoms, diminishment of extent of disease, stabilization (i.e., not worsening) of disease, preventing spread (i.e., metastasis) of disease, delaying or slowing of disease progression, amelioration or palliation of the disease state, and remission (partial or total) whether detectable or undetectable. In addition, “treat”, “treating”, and “treatment” can also mean prolonging survival as compared to expected survival if not receiving treatment.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Pro drug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs-principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000). Rationale and applications of lipids as prodrug carriers, Eur. J Pharm. Sci., 11 Suppl 2:S15-27; Wang, W. et al. (1999). Prodrug approaches to the improved delivery of peptide drugs. Curr. Pharm. Des., 5(4):265-87.

As used herein, the term “topically active agents” refers to compositions of the present disclosure that elicit pharmacological responses at the site of application (contact) to a host.

As used herein, the term “topically” refers to application of the compositions of the present disclosure to the surface of the skin and mucosal cells and tissues.

The terms “alk” or “alkyl” refer to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, and the like. Lower alkyl groups, that is, alkyl groups of 1 to 6 carbon atoms, are generally most preferred. The term “substituted alkyl” refers to alkyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally subsituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The term “alkoxy” means an alkyl group linked to oxygen thus: R—O—. In this function, R represents the alkyl group. An example would be the methoxy group CH₃O—.

The terms “ar” or “aryl” refer to aromatic homocyclic (i.e., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, etc., where optionally one or more pair of substituents together with the atoms to which they are bonded form a 3 to 7 member ring.

The term “aminoacyl” refer to groups having an C₁₋₆ acyl (alkanoyl) group attached to an amino nitrogen, as well as to groups having an arylsubstituted C₂₋₆ substituted acyl group attached to an amino nitrogen.

The term “alkylamino” groups and “dialkylamino” refer to groups having a C₁₋₆ alkyl or dialkyl group attached to an amino nitrogen, respectively, as well as to groups having an alkyl or dialkyl substituted C₁₋₆ alkyl or dialkyl group attached to an amino nitrogen, respectively.

The terms “halogen” and “halo” refer to fluorine, chlorine, bromine and iodine.

The terms “heterocycle”, “heterocyclic”, “heterocyclic group” or “heterocyclo” refer to fully saturated or partially or completely unsaturated, including aromatic (“heteroaryl”) or nonaromatic cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be either fused, bridged and/or joined through one or more spiro unions.

The terms “substituted heterocycle”, “substituted heterocyclic”, “substituted heterocyclic group” and “substituted heterocyclo” refer to heterocycle, heterocyclic and heterocyclo groups substituted with one or more groups preferably selected from alkyl, substituted alkyl, alkenyl, oxo, aryl, substituted aryl, heterocyclo, substituted heterocyclo, carbocyclo (optionally substituted), halo, hydroxy, alkoxy (optionally substituted), aryloxy (optionally substituted), alkanoyl (optionally substituted), aroyl (optionally substituted), alkylester (optionally substituted), arylester (optionally substituted), cyano, nitro, amido, amino, substituted amino, lactam, urea, urethane, sulfonyl, and the like, where optionally one or more pair of substituents together with the atoms to which they are bonded form a 3 to 7 member ring.

Throughout the specification, groups and substituents thereof may be chosen to provide stable moieties and compounds.

The disclosed compounds may form salts that are also within the scope of this disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when a compound having a certain formula contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful (e.g., in isolation or purification steps which may be employed during preparation). Salts of the compounds having a certain formula may be formed, for example, by reacting of a first compound with an amount of acid or base, such as an equivalent amount, in a medium such as one in which the salt precipitates or in an aqueous medium followed by lyophilization.

The disclosed compounds that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The disclosed compounds that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (e.g., organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

To the extent that the disclosed compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected stereoisomers. The chiral centers of the compounds of the present disclosure can have the S or R configuration as defined by the IUPAC 1974 Recommendations.

The terms “including”, “such as”, “for example” and the like are intended to refer to exemplary embodiments and not to limit the scope of the present disclosure.

Hypoxia Inducible Factor (HIF-1)

HIF-1 is a primary transcriptional factor responsible for specific induction of genes in hypoxia. HIF-1 is has of two sub-units belonging to the bHBH-PAS family: HIF-1α and aryl hydrocarbon receptor nuclear translocator (ARNT, also known as HIF-1β). To activate transaction of target genes, HIF-1α dimerizes with HIF-1β and binds to consensus sequences (hypoxia responsive element, HRE) in the promoter or enhancer regions of these genes. Proteins encoded by such genes include vascular endothelial growth factor (VEGF), erythropoietin, glucose transporter-i, glycolytic enzymes and tyrosine hydroxylase (Semenza G. L. Regulation of mammalian of homeostasis by hypoxia-inducible factor 1. Annu Rev Cell Dev Biol 15, 551-78 (1999)).

In normoxia, von Hippel Lindau protein (pVHL) organizes the assembly of a complex that activates the E3 ubiquitin ligase, which then ubiquitinylates HIF-1α, targeting its degradation. The interaction between HIF-1α and pVHL is regulated through hydroxylation of two proline residues of HIF-1α by a prolyl hydroxylase. In the absence of oxygen, this enzyme is no longer active and HIF-1α does not interact with pVHL and accumulates intracellularly (Ivan, M. et al. HIFα targeted for VHL-mediated destruction by proline hydroxylation: implications for O₂ sensing. Science 292, 464-8 (2001); Jaakkola, P. et al. Targeting of HIFα to the von Hipplel Lindau ubiquitylation complex by O₂ regulated prolyl hydroxylation. Science 292, 468-72 (2001)).

Tumor hypoxia increases malignant progression and metastasis by promoting angiogenesis through the induction of proangiogenic proteins such as VEGF (Schweiki, D. et al. Vascular endothelial growth factor induced by hypoxia may mediate hypoxia-induced angiogenesis. Nature 359, 843-5 (1992)). Since most genes induced by hypoxia are regulated by HIF-1α, this protein plays a pivotal role in tumor development (Dachs G. U. and Chaplin, D. J. Microenvironmental control of gene expression: implications for tumor angiogenesis, progression, and metastasis. Semin Radiat Oncol 8, 208-16 (1998); Maxwell, P. H. et al. Hypoxia-inducible factor-1 mediates gene expression in solid tumors and influences both angiogenesis and tumor growth. Proc Natl Acad Sci USA 94, 8104-9 (1997); Semenza, G. L. Hypoxia-inducible factor 1: master regulator of O₂ homeostasis. Curr Opin Genet Dev 8, 588-94 (1998)).

Histological analyses have shown that an increased level of intracellular HIF-1α was associated with poor prognosis and resistance to therapy in head and neck, breast, cervical, and oropharyngeal cancers (Beasley, N. J. P. et al. Hypoxia-inducible factors HIF-1α and HIF-2α in head and neck cancer: relationship to tumor biology and treatment outcome in surgically resected patients, Cancer Res 62, 2493-7 (2002); Schindl, M. et al. Overexpresssion of hypoxia-inducible factor la is associated with an unfavorable prognosis in lymph node-positive breast cancer, Clin Cancer Res 8, 1831-7(2002); Birner, P. et al. Overexpression of hypoxia-inducible factor 1a is a marker for an unfavorable prognosis in early-stage invasive cervical cancer, Cancer Res 60, 4693-6 (2000); Aebersold, D. M. et al. Expression of hypoxia-inducible factor-1α: a novel predictive and prognostic parameter in the radiotherapy of oropharyngeal cancer, Cancer Res 61, 2911-6 (2001)). HIF-1α was overexpressed in the cytoplasm and the nucleus of colon, breast, gastric, lung, skin, ovarian, pancreatic, prostate and renal carcinomas.

Additional details regarding HIF-1 are described in Examples 1 below.

HIF-1 Inhibitor Compositions

In general, the HIF-1 inhibitor compositions can be used to treat and/or prevent cancers or tumors, and cancer related conditions in a host; interfere, inhibit, and/or block signal transduction through the HIF-1 pathway; and treat and/or prevent hypoxia-related pathologies, for example. Additional uses and/or applications of the HIF-1 inhibitor compositions are described below.

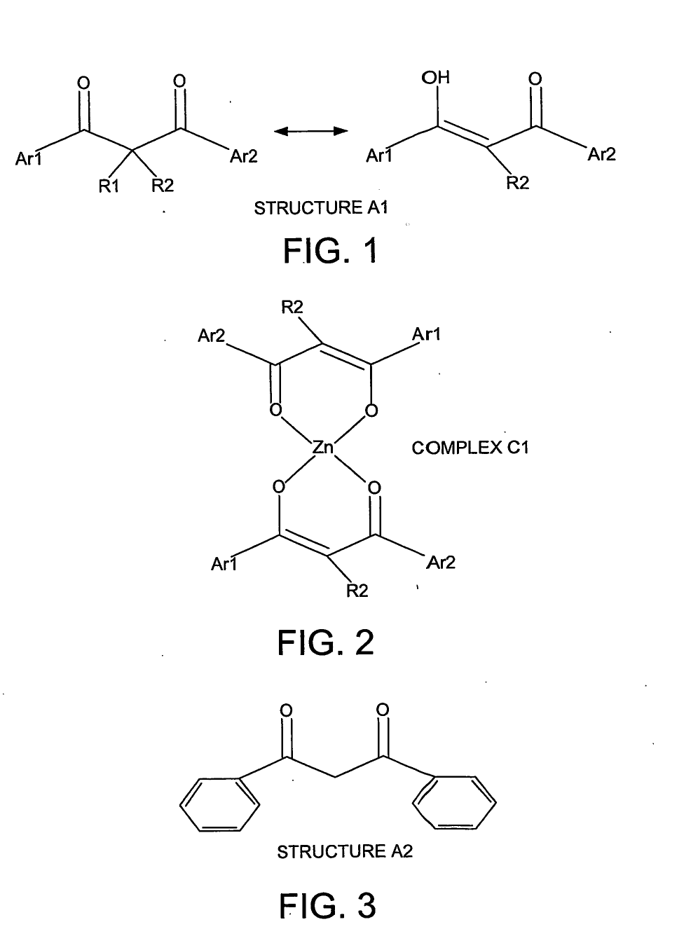

An embodiment of the HIF-1 inhibitor compositions can include, but is not limited to, a formulation including a beta-diketone compound (e.g., structure A1 in FIG. 1 (aryl derivatives of the beta-diketone compound)) and a zinc compound (e.g., ZnCl₂). The two components can be used individually, in combination, or as bidentate zinc chelates (e.g., FIG. 2). An embodiment of a beta-diketone compound, dibenzoylmethane, is shown in FIG. 3 and is termed structure A2.

Where such forms exist, beta-diketones can include beta-diketone analogues, beta-diketone compound homologues, beta-diketone compound isomers, or beta-diketone derivatives thereof, that can function in a similar biological manner as beta-diketones to treat and/or prevent cancers or tumors, and cancer related conditions in a host; interfere, inhibit, and/or block signal transduction through the HIF-1 pathway; and treat and/or prevent hypoxia-related pathologies. In addition, where such forms exist, beta-diketones can include pharmaceutically acceptable salts, esters, and prodrugs of the beta-diketones described or referred to herein.

In particular beta-diketones can include, but are not limited to, dibenzoylmethane-type compounds, where such forms exist, and their respective analogues, homologues, isomers, and derivatives.

Where such forms exist, dibenzoylmethane-type compounds can include, but are not limited to, dibenzoylmethane derivatives that function to treat and/or prevent cancers or tumors, and cancer related conditions in a host; interfere, inhibit, and/or block signal transduction through the HIF-1 pathway; and treat and/or prevent hypoxia-related pathologies. In addition, where such forms exist, dibenzoylmethane-type compounds can include pharmaceutically acceptable salts, esters, and prodrugs of the dibenzoylmethane-type compounds described or referred to herein.

In another embodiment, the HIF-1 inhibitor composition can include, but is not limited to, beta-diketone bidentate zinc chelate compounds as shown in FIG. 2 (complex C1). Although not intending to be bound by theory, the beta-diketone compound can be reacted with a zinc compound (e.g., ZnCl₂) to produce a beta-diketone bidentate zinc chelate compound.

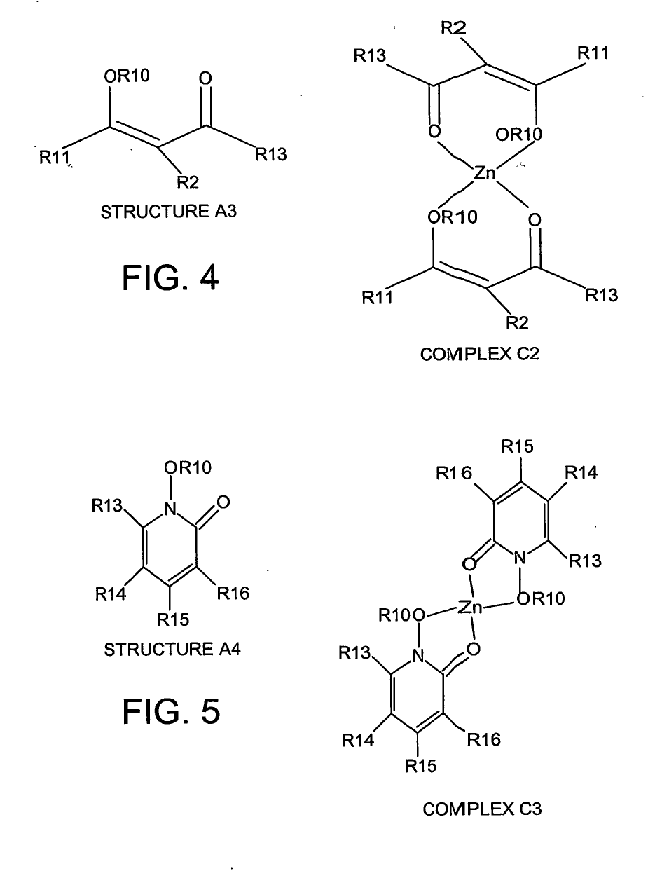

In another embodiment, the HIF-1 inhibitor composition can include, but is not limited to, a formulation including structure A3 (e.g., derivatives of the enol form of dibenzoylmethane) in FIG. 4 and a zinc compound (e.g., ZnCl₂). The two components can be administered individually or in combination. In another embodiment, the HIF-1 inhibitor composition can include, but is not limited to, a bidentate zinc chelate as shown in FIG. 4 (complex C2). Although not intending to be bound by theory, the compound having structure A3 can be reacted with a zinc compound to produce the bidentate zinc chelate.

In another embodiment, the HIF-1 inhibitor composition can include, but is not limited to, a formulation including structure A4 (e.g., 1-hydroxy-2-pyridone and derivatives) in FIG. 5 and a zinc compound (e.g., ZnCl₂). The two components can be administered individually or in combination. In another embodiment, the HIF-1 inhibitor composition can include, but is not limited to, a bidentate zinc chelate as shown in FIG. 5 (complex C3). Although not intending to be bound by theory, the compound having structure A4 can be reacted with a zinc compound to produce the bidentate zinc chelate.

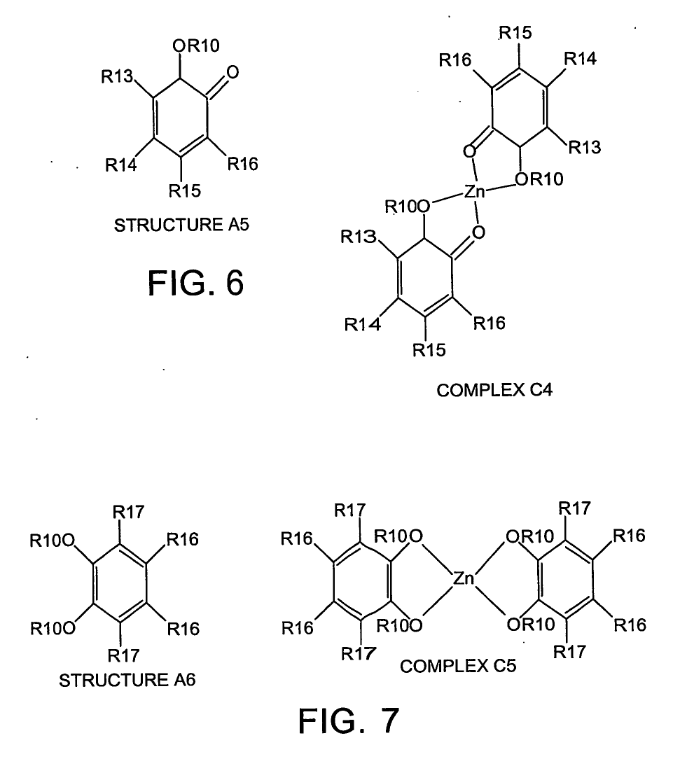

In another embodiment, the HIF-1 inhibitor composition can include, but is not limited to, a formulation including structure A5 (e.g., 2-hydroxy-3,5-cyclohexadien-1-one and derivatives) in FIG. 6 and a zinc compound (e.g., ZnCl₂). The two components can be administered individually or in combination. In another embodiment, the HIF-1 inhibitor composition can include, but is not limited to, a bidentate zinc chelate as shown in FIG. 6 (complex C4). Although not intending to be bound by theory, the compound having structure A5 can be reacted with a zinc compound to produce the bidentate zinc chelate.

In another embodiment, the HIF-1 inhibitor composition can include, but is not limited to, a formulation including structure A6 (e.g., l-hydroxy-2-alkyloxy-benzene and derivatives) in FIG. 7 and a zinc compound (e.g., ZnCl₂). The two components can be administered individually or in combination. In another embodiment, the HIF-1 inhibitor composition can include, but is not limited to, a bidentate zinc chelate as shown in FIG. 7 (complex C5). Although not intending to be bound by theory, the compound having structure A6 can be reacted with a zinc compound to produce the bidentate zinc chelate.

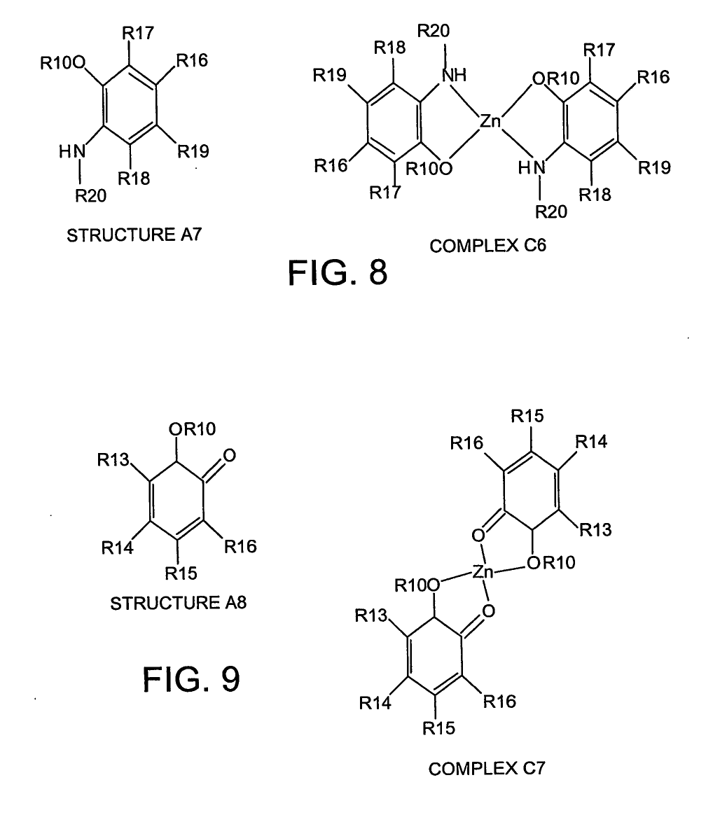

In another embodiment, the HIF-1 inhibitor composition can include, but is not limited to, a formulation including structure A7 (e.g., 1-amino-2-alkyloxy-benzene and derivatives) in FIG. 8 and a zinc compound (e.g., ZnCl₂). The two components can be administered individually or in combination. In another embodiment, the HIF-1 inhibitor composition can include, but is not limited to, a bidentate zinc chelate as shown in FIG. 8 (complex C6). Although not intending to be bound by theory, the compound having structure A7 can be reacted with a zinc compound to produce the bidentate zinc chelate.

In another embodiment, the HIF-1 inhibitor composition can include, but is not limited to, a formulation including structure A8 (e.g., 2-hydroxymethylfuran and derivatives) in FIG. 9 and a zinc compound (e.g., ZnCl₂). The two components can be administered individually or in combination. In another embodiment, the HIF-1 inhibitor composition can include, but is not limited to, a bidentate zinc chelate as shown in FIG. 9 (complex C7). Although not intending to be bound by theory, the compound having structure A8 can be reacted with a zinc compound to produce the bidentate zinc chelate.

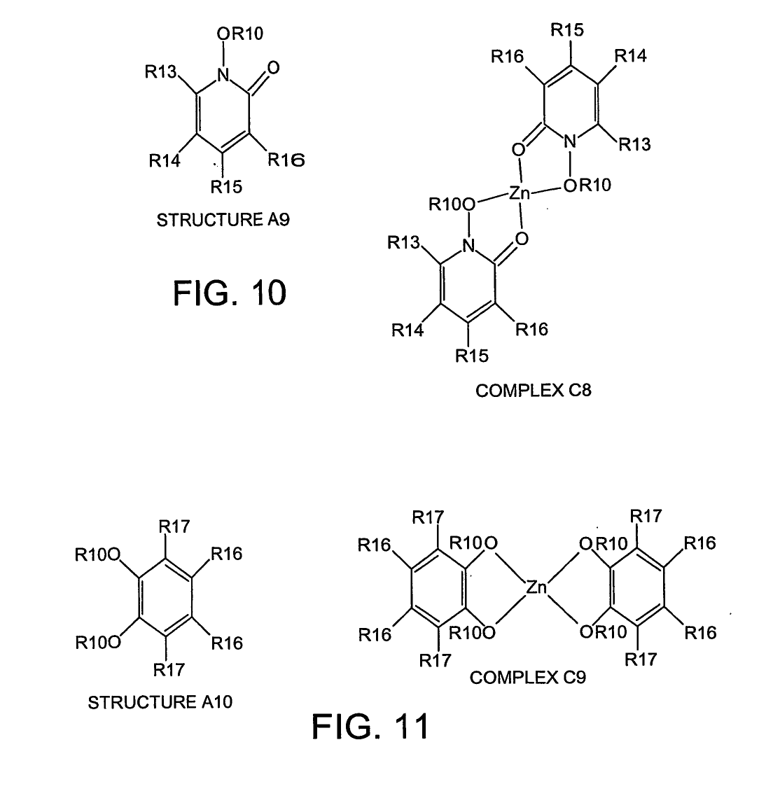

In another embodiment, the HIF-1 inhibitor composition can include, but is not limited to, a formulation including structure A9 (e.g., 2-aminomethylfuran and derivatives) in FIG. 10 and a zinc compound (e.g., ZnCl₂). The two components can be administered individually or in combination. In another embodiment, the HIF-1 inhibitor composition can include, but is not limited to, a bidentate zinc chelate as shown in FIG. 10 (complex C8). Although not intending to be bound by theory, the compound having structure A9 can be reacted with a zinc compound to produce the bidentate zinc chelate.

In another embodiment, the HIF-1 inhibitor composition can include, but is not limited to, a formulation including structure A10 (e.g., 3-hydroxy-2-furanone and derivatives) in FIG. 11 and a zinc compound (e.g., ZnCl₂). The two components can be administered individually or in combination. In another embodiment, the HIF-1 inhibitor composition can include, but is not limited to, a bidentate zinc chelate as shown in FIG. 11 (complex C9). Although not intending to be bound by theory, the compound having structure A10 can be reacted with a zinc compound to produce the bidentate zinc chelate.

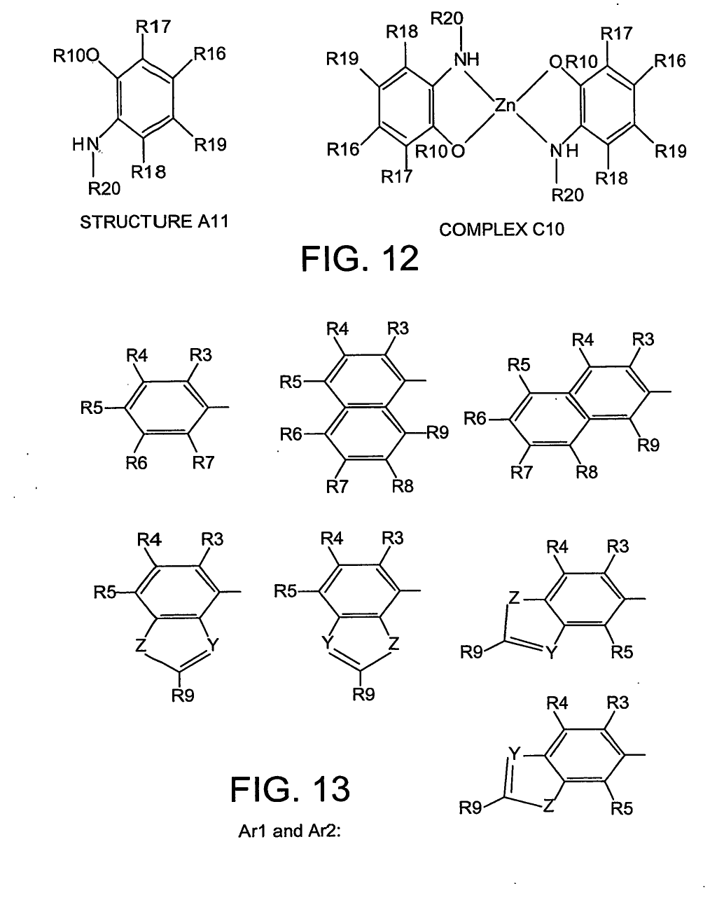

In another embodiment, the HIF-1 inhibitor composition can include, but is not limited to, a formulation including structure A11 (e.g., 3-amino-2-furanone and derivatives) in FIG. 12 and a zinc compound (e.g., ZnCl₂). The two components can be administered individually or in combination. In another embodiment, the HIF-1 inhibitor composition can include, but is not limited to, a bidentate zinc chelate as shown in FIG. 12 (complex C10). Although not intending to be bound by theory, the compound having structure A11 can be reacted with a zinc compound to produce the bidentate zinc chelate.

Exemplary functional groups of the dibenzoylmethane-type compounds are indicated as Ar1, Ar2, R1, and R2. FIG. 13 illustrates exemplary functional groups (e.g., cyclic hydrocarbons and heterocyclic hydrocarbons) Ar1 and Ar2, which can include functional groups R3, R4, R5, R6, R7, R8, R9, R10, R11, R12, R13, R14, R15, R16, R17, R18, R19, R20, Y, and Z. The functional groups R1, R2, R3, R4, R5, R6, R7, R8, R9, R11, R12, R13, R14, R15, R16, R17, R18, and R19, can each individually be a functional group selected from, but not limited to, hydrogen, alkyl groups, aryl groups, halo groups (F, Cl, Br, and I) hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons. The functional groups R10 and R20 can each individually be a functional group selected from, but not limited to, hydrogen and a sulfonyl group. The functional group Y includes, but is not limited to, nitrogen and CR1, while Z includes, but is not limited to, oxygen, sulphur, NR1, and CR1.

The zinc compound can be a compound such as, but not limited to, ZnCl₂, ZnSO₄, and combinations thereof.

Methods of Use

Some embodiments of the present disclosure are directed to interfering, inhibiting, or blocking signal transduction through the HIF-1 pathway. Such inhibition can be accomplished by binding of HIF-1 or molecules associated with HIF-1 with the HIF-1 inhibitor compositions described herein or their derivatives, pharmaceutically acceptable salts, prodrugs, etc., and combinations thereof (hereinafter “HIF-1 inhibitor composition”) to render HIF-1 inactive or unavailable. Alternatively, the HIF-1 pathway can be inhibited, in whole or in part, by preventing the expression of HIF-1 in a cell (e.g., through preventing HIF mRNA transcription, post-transcriptional modification of HIF mRNA, translation of HIF mRNA, posttranslational modification of HIF protein and HIF stability) with the HIF-1 inhibitor compositions. HIF-1 inhibition can also be achieved by interfering with the binding of HIF-1 or HIF-1 complexes to the hypoxia responsive element with the HIF-1 inhibitor compositions.

One embodiment provides a method for the treatment or prevention of a hypoxia-related pathology by administering to a host (e.g., a mammal) in need of such treatment, an HIF-1 inhibiting amount of the HIF-1 inhibitor composition.

Another embodiment provides a method of modulating HIF-1 activity in a cell (e.g., an eukaryotic cell) by contacting the cell with an HIF-1 inhibiting amount of the HIF-1 inhibitor composition.

Still another embodiment provides a method of treating or preventing cancer and/or a tumor in a host by administering to the host a HIF-1 inhibiting amount of the HIF-1 inhibitor composition.

As mentioned above, embodiments of the HIF-1 inhibitor composition can be used to treat cancers, tumors, and related pathologies. In this regard, the term “cancer” is a general term for diseases in which abnormal cells divide without control. Cancer cells can invade nearby tissues and can spread through the bloodstream and lymphatic system to other parts of the body. It has been discovered that the administration of an HIF-1 inhibitor composition to a host (e.g., a mammal) inhibits and/or reduces cancer, tumor growth or formation, the metastasis of tumor cells, and the like.

There are several main types of cancer, and the HIF-1 inhibitor composition can be used to treat any type of cancer. For example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. The compositions described herein can be used to reduce, inhibit, or diminish the proliferation of tumor cells, and thereby assist in reducing the size of a tumor. In particular, the disclosed compositions are useful for the treatment of solid tumors or pathologies in areas of hypoxia. Cancers can also have genetic alterations that lead to constitutive HIF expression independently of hypoxia.

Representative cancers that may treated with the the HIF-1 inhibitor composition and methods include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth) and continue their rapid growth and division in this new location. This is called metastasis. Once malignant cells have metastasized, achieving cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain. The HIF-1 inhibitor composition provided herein can be used to treat benign or malignant tumors.

Accordingly, one embodiment provides a method of modulating gene transcription, for example the transcription of VEGF, erythropoietin, glucose transporter-1, glycolytic enzymes, or tyrosine hydroxylase, in a cell (e.g., a tumor or cancer cell) by contacting the cell with an HIF-1 inhibiting amount of one or more of the HIF-1 inhibitor compositions. Alternatively, such transcription can be inhibited in a host by administering to the host an HIF-1 inhibiting amount of the HIF-1 inhibitor composition.

Another embodiment provides a method of modulating gene expression in a tumor cell by contacting the tumor cell with an HIF-1 modulating amount of one or more of the HIF-1 inhibitor composition. The modulation of the HIF-1 pathway with the disclosed compounds and compositions can occur at transcriptional, translational and/or post-translational levels. The disclosed compounds can modulate gene transcriptions by binding to HIF-1 and preventing HIF-1 from forming complexes with other molecules including DNA and proteins. For example, the HIF-1 inhibitor composition can bind to HIF-1 and induce conformational changes that prevent HIF-1 from interacting with its biological targets. Alternatively, the HIF-1 inhibitor composition can bind HIF-1 and form aggresomes or other complexes that sequester HIF-1 or otherwise physically prevent HIF-1 from interacting with other biological molecules. In addition, the HIF-1 inhibitor composition can inhibit or interfere with the intracellular transport of HIF-1 including, but not limited to, the translocation of HIF-1 from the cytoplasm to the nucleus.

Another embodiment provides a method for treating a hypoxia-related pathology by administering the combination of the HIF-1 inhibitor composition with conventional chemotherapeutic agents and/or radiotherapy. For example, the HIF-1 inhibitor composition can be used to treat a pathology, for example a proliferative pathology such as cancer or other hypoxia related pathology independently or in combination with one another or with one or more additional therapeutic agents. Representative therapeutic agents include but are not limited to antibiotics, anti-inflammatories, anti-oxidants, analgesics, radioisotopes, chemotherapeutic agents and targeted therapeutic agents such as nascopine, paclitaxel, nocodazole, vinca alkaloids, adriamycin, alkeran, Ara-C, BiCNU, busulfan, CCNU, carboplatinum, cisplatinum, cytoxan, daunorubicin, DTIC, 5-FU, fludarabine, hydrea, idarubicin, ifosfamide, methotrexate, mithramycin, mitomycin, mitoxantrone, nitrogen, mustard, velban, vincristine, VP-16, gemcitabine (gemzar), herceptin, irinotecan, (camptosar, CPT-11), leustatin, navelbine, rituxan, STI-571, taxotere, topotecan, (hycamtin), xeloda (capecitabine), zevelin, and combinations thereof.

It will be appreciated that the HIF-1 inhibitor composition can be used in combination with radiation therapy or surgical procedures for the treatment of a pathology (e.g., cancers and/or a tumors).

In another embodiment, the HIF-1 inhibitor compositions are administered to a host having developed resistance to chemotherapeutic agents.

Pharmaceutical Compositions

Pharmaceutical compositions and dosage forms include a pharmaceutically acceptable salt of disclosed or a pharmaceutically acceptable polymorph, solvate, hydrate, dehydrate, co-crystal, anhydrous, or amorphous form thereof.

Pharmaceutical compositions and unit dosage forms typically also include one or more pharmaceutically acceptable excipients or diluents. Advantages provided by the HIF-1 inhibitor composition, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the HIF-1 inhibitor composition are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the HIF-1 inhibitor composition can vary depending on their use. For example, a dosage form used in the acute treatment of a disease or disorder may contain larger amounts of the active ingredient (e.g., the HIF-1 inhibitor composition) than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. (e.g., Remington's Pharmaceutical Sciences, 18th ed., Mack Publishing, Easton, Pa. (1990)).

Typical pharmaceutical compositions and dosage forms can include one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that include primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure include a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, and more preferably in an amount of from 50 mg to 500 mg.

Additionally, the HIF-1 inhibitor composition can be delivered using lipid- or polymer-based nanoparticles. For example, the nanoparticles can be designed to improve the pharmacological and therapeutic properties of drugs administered parenterally (Allen, T. M., Cullis, P. R. Drug delivery systems: entering the mainstream. Science. 303(5665): 1818-22 (2004)).

Oral Dosage Forms

Pharmaceutical compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the HIF-1 inhibitor composition, and may be prepared by methods of pharmacy well known to those skilled in the art. (Remington's Pharmaceutical Sciences, 18th ed., Mack Publishing, Easton, Pa. (1990)).

Typical oral dosage forms of the HIF-1 inhibitor composition are prepared by combining the pharmaceutically acceptable salt of the HIF-1 inhibitor composition in an intimate admixture with at least one excipient according to conventional pharmaceutical compounding techniques. Excipients can take a wide variety of forms depending on the form of the HIF-1 inhibitor composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the HIF-1 inhibitor composition to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions include from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can include excipients that are well known in the art and are listed in the USP (XXI)/NF (XVI), which is incorporated herein by reference. In general, lactose-free compositions include a pharmaceutically acceptable salt of the HIF-1 inhibitor composition, a binder/filler, and a lubricant in pharmaceutically compatible and pharmaceutically acceptable amounts. Preferred lactose-free dosage forms include a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. (e.g., Jens T. Carstensen, Drug Stability: Principles & Practice, 379-80 (2nd ed., Marcel Dekker, NY, N.Y.: 1995)). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that include lactose and at least one active ingredient that includes a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms

Pharmaceutically acceptable salts of the disclosed compounds can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) Minimization of drug accumulation; 7) reduction in blood level fluctuations; 8) improvement in efficacy of treatment; 9) reduction of potentiation or loss of drug activity; and 10) improvement in speed of control of diseases or conditions. (e.g., Kim, Cherng-ju, Controlled Release Dosage Form Design, 2 (Technomic Publishing, Lancaster, Pa.: 2000)).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (e.g., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder (e.g., a HIF-1 inhibitor composition) into a hard tablet, coating the tablet with cellulose derivatives to form a semi-permeable membrane, and then drilling an orifice in the coating (e.g., with a laser). (e.g., Kim, Cherng-ju, Controlled Release Dosage Form Design, 231-238 (Technomic Publishing, Lancaster, Pa.: 2000)). The advantage of such dosage forms is that the delivery rate of the drug is not influenced by physiological or experimental conditions. Even a drug with a pH-dependent solubility can be delivered at a constant rate regardless of the pH of the delivery medium.

A specific dosage form of the HIF-1 inhibitor composition includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes a salt of an HIF-1 inhibitor composition, or a polymorph, solvate, hydrate, dehydrate, co-crystal, anhydrous, or amorphous form thereof. (e.g., U.S. Pat. No. 6,368,626, the entirety of which is incorporated herein by reference).

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation includes a salt of a HIF-1 inhibitor composition, or a polymorph, solvate, hydrate, dehydrate, co-crystal, anhydrous, or amorphous form thereof (e.g., U.S. Pat. No. 6,342,249, the entirety of which is incorporated herein by reference).

Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a HIF-1 inhibitor composition disclosed herein can also be incorporated into the parenteral dosage forms of the disclosure, including conventional and controlled-release parenteral dosage forms.

Topical, Transdermal and Mucosal Dosage Forms

Topical dosage forms of the disclosure include, but are not limited to, creams, lotions, ointments, gels, shampoos, sprays, aerosols, solutions, emulsions, and other forms know to one of skill in the art. (e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985)). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. (e.g., Remington's Pharmaceutical Sciences, 18.sup.th Ed., Mack Publishing, Easton, Pa. (1990)).

Transdermal and mucosal dosage forms of the HIF-1 inhibitor composition include, but are not limited to, ophthalmic solutions, patches, sprays, aerosols, creams, lotions, suppositories, ointments, gels, solutions, emulsions, suspensions, or other forms known to one of skill in the art. (e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985)). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of an the HIF-1 inhibitor composition, For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of an HIF-1 inhibitor composition can be used to further adjust the properties of the resulting composition.

Kits

Typically, active ingredients of the pharmaceutical compositions of the disclosure are preferably not administered to a patient at the same time or by the same route of administration. This disclosure therefore encompasses kits which, when used by the medical practitioner, can simplify the administration of appropriate amounts of active ingredients to a patient.

A typical kit includes a unit dosage form of a pharmaceutically acceptable salt of an HIF-1 inhibitor composition and optionally, a unit dosage form of a second pharmacologically active compound, such as anti-proliferative agent, or anti-cancer agent. In particular, the pharmaceutically acceptable salt of an HIF-1 inhibitor composition is the sodium, lithium, or potassium salt, or a polymorph, solvate, hydrate, dehydrate, co-crystal, anhydrous, or amorphous form thereof. A kit may further include a device that can be used to administer the active ingredient. Examples of such devices include, but are not limited to, syringes, drip bags, patches, and inhalers.

Kits of the disclosure can further include pharmaceutically acceptable vehicles that can be used to administer one or more active ingredients (e.g, an HIF-1 inhibitor composition). For example, if an active ingredient is provided in a solid form that must be reconstituted for parenteral administration, the kit can include a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Other embodiments are directed to the use of the HIF-1 inhibitor composition in the preparation of a medicament for the treatment hypoxia-related pathology.

### EXAMPLE 1

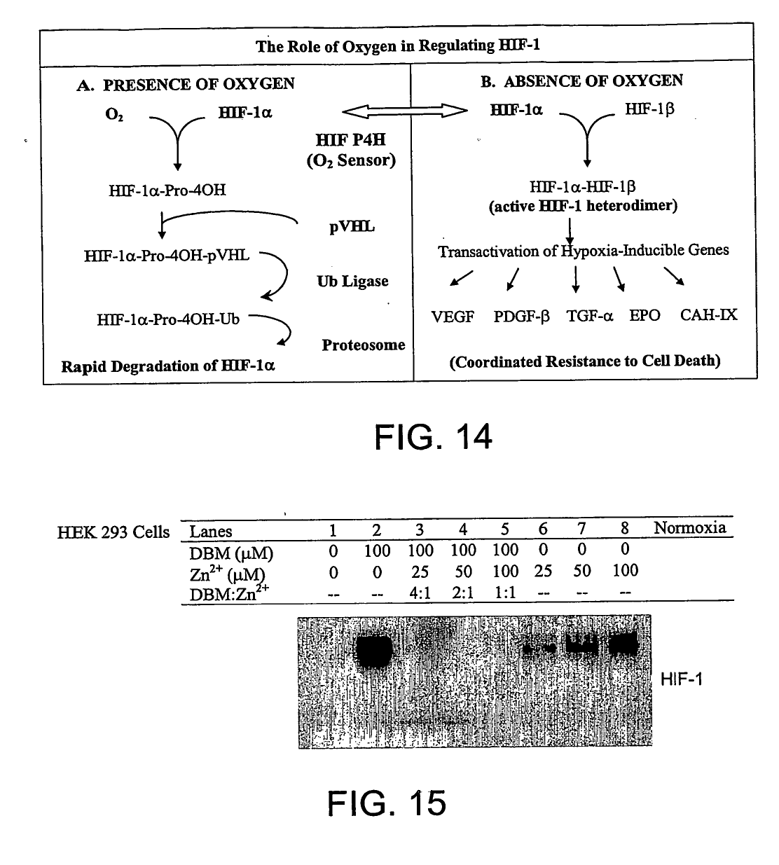

Hypoxia inducible factor-1 is a nuclear transcription factor composed of α and β subunits. Both subunits are synthesized constituitively and have no independent activity. HIF-1α regulates the activity of the HIF-1 heterodimer by undergoing rapid proteosomal degradation in the presence of ambient O₂ as shown, for example, in FIG. 14 (A). The HIF prolyl-4-hydroxylase (P4H) converts Pro⁴⁰² and Pro⁵⁶⁴ of HIF-1α to 4-hydroxyproline (Pro-4OH) thereby allowing these residues to serve as binding sites for the von Hippel Lindau gene product (pVHL), a prerequisite for binding to E3 ubiquitin ligase (Ub Ligase). Ub Ligase attaches polyubiquitin chains to the Pro-4OH residues so as to target HIF-1α for proteosomal degradation [1-6]. Degradation of HIF-1α is markedly reduced under hypoxic conditions since ambient O₂ is a substrate for the prolyl 4-hydroxylation reaction making HIP P4H an efficient O₂ sensor. Acetylation of Lys532 is also required for VHL-mediated proteosomal degradation of HIF-1α, though the level of acetylation gradually decreases with increased length of exposure to hypoxia [7]. In addition, transactivation of HIF-1α is regulated by hydroxylation of Asn803 [8].

Hypoxia is not a normal physiologic state, though it is a common feature of many neoplasms. Hypoxia causes HIF-1α to accumulate and bind to HIF-1β so that active HIF-1 is formed as shown, for example, in FIG. 14 (B). HIF-1 mediates the transactivation of vascular endothelial growth factor (VEGF), platelet derived growth factor-β (PDGF-β), tumor growth factor-α (TGF-α), erythropoietin (EPO), carbonic anhydrase IX (CAH-IX), and more than 60 other genes. Activation of these genes provides hypoxic tumors redundant and complementary mechanisms for survival and resistance to cancer therapy.

Mutations that inactivate the VHL gene occur in 50% or more of sporadic kidney cancers and are the cause of hereditary clear cell renal cell carcinoma. The resulting accumulation of HIF-1α initiates and sustains carcinogenesis with no need for hypoxia. This primary carcinogenic function in kidney cancer stands in sharp contrast to the secondary role of HIF-1α in stimulating the growth of other forms of cancer in response to hypoxia.

More than 30,000 new cases of kidney cancer are diagnosed each year. Metastatic kidney cancer is incurable in the vast majority of cases with a median survival of about 12 to 18 months. Kidney cancer does not respond well to traditional chemotherapeutic agents or radiation. Standard treatment with immunotherapy such as interleukin-2 or interferon-α is very expensive, largely ineffective and is associated with significant clinical toxicity. Novel approaches are desperately needed for treating kidney cancer.

Cancers of the kidney tend to be highly vascular with areas of spontaneous necrosis and hypoxia. Clinical responses have been observed in patients with metastatic disease when treated with high doses of bevacizumab (Avastin), a humanized monoclonal antibody specific for VEGF [9]. Another recent study reported that the combination of bevacizumab with erlotinib (Tarceva) could have greater activity against kidney cancer than any therapy reported to date [10]. HIF-1α can be stabilized by signals from the epidermal growth factor receptor (EGFR), signals that are blocked by erlotinib. A growing body of evidence suggests that blocking targets upstream (EGFR) and downstream (VEGF) from HIF-1α produce clinical responses in patients with metastatic kidney cancer. One may infer that potent anti-neoplastic effects would result from down-regulating HIF-1α in patients with kidney cancer. Taken together, these data provide a strong biological rationale for targeting HIF-1α in kidney cancer.

HIF-1α is overexpressed in many cancers [11], and a variety of strategies are being pursued to develop it as a new therapeutic target [11-19]. It has been previously shown that HIF-1α and VEGF levels rapidly increase in LNCaP human prostate cancer cells treated with either DBM (1,3-diphenylpropane-1,3-dione) or cobalt (Co²⁺) under conditions of ambient O₂ [20]. DBM is a naturally occurring bidentate iron chelator found in certain species of licorice plant. These data are consistent with prior reports that iron chelators and di- or trivalent metals inhibit HIF P4H, presumably by binding or competing with Fe²⁺ in the enzymatic active site.

In subsequent studies, similar data has been found for HEK 293 human embryonic kidney cells and HT144 melanoma cells treated with either DBM or Zn²⁺. Surprisingly, however, no HIF-1α was detected in either cell line when treated simultaneously with DBM plus Zn²⁺. This finding appears to be unique to the combination of DBM with Zn²⁺ since it was not observed in cells exposed to DBM plus other metals such as Fe³⁺, Co²⁺, Cd²⁺, and Ni²⁺. Furthermore, this unexpected reversal of the independent effects of DBM and Zn²⁺ was not blocked by hypoxia. These observations are counterintuitive since inhibitors of HIF P4H should impede degradation of HIF-1α. Also, these two HIF P4H inhibitors appear to work in concert under inhibitory conditions to down-regulate HIF-1α with little effect on HIF-1α mRNA levels. Moreover, down-regulation of HIF-1α by DBM plus Zn²⁺ does not require pVHL or the 26S proteosome since it was observed in VHL (−/−) RCC-4 renal cell carcinoma cells treated with MG-132, a proteosome inhibitor. The corresponding experimental data are presented in FIGS. 15 through 19.

The first unexpected finding was that the combination of DBM plus Zn²⁺ reversed the induced stabilization of HIF-1α observed when the cells were exposed to either DBM or Zn²⁺ alone under normoxic conditions (FIG. 15). HEK 293 embryonic kidney cells were incubated at ambient levels of oxygen for 1 hr with the addition of DBM alone, Zn²⁺ alone or the combination. HIF-1α was not detected at baseline by Western blot (FIG. 15, lane 1). The addition of DBM alone at 100 μM resulted in a significant increase in HIF-1α levels (FIG. 15, lane 2). The addition of Zn²⁺ alone also increased HIF-1α levels in a dose dependent manner at concentrations of 25 μM to 100 μM (FIG. 15, lanes 6, 7, and 8). Surprisingly, no HIF-1α was detected when the cells were treated simultaneously with DBM plus Zn²⁺ (FIG. 15, lanes 3, 4, and 5).

One possible explanation for the loss of HIF-1α stabilization in cells treated with DBM plus Zn²⁺ is chelation. DBM is a bidentate metal chelator that could itself bind to Zn²⁺ in a 2:1 stochiometric ratio, however, the observed loss of HIF-1α stabilization occurred when either DBM or Zn was present in 50 μM excess at ratios of 4:1 (FIG. 15, lane 3) and 1:1 (FIG. 15, lane 5), respectively. Therefore, chelation alone does not appear to be a viable explanation for these data. Presumably, the increases in HIF-1α in FIG. 15 resulted from HIF P4H inhibition leading to diminished prolyl-4-hydroxylation and disruption of proteosomal degradation.

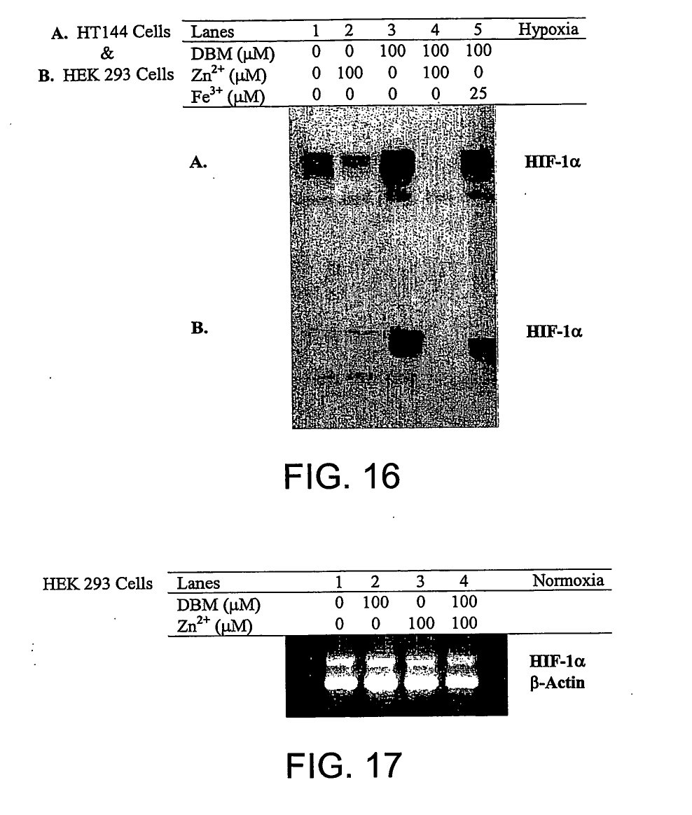

The next important observation was that hypoxia did not counteract the effect of DBM plus Zn²⁺ on HIF-1α levels (FIG. 16). HT 144 melanoma cells (FIG. 16 (A)) and HEK 293 embryonic kidney cells (FIG. 16 (B)) were incubated for 2 hours under 1% O₂. HIF-1α was detected by Western blot at baseline (FIGS. 15 (A) and (B) lane 1). The addition of Zn²⁺ alone decreased the more rapidly migrating isoforms of HIF-1α in HT 144 cells (FIG. 16 (A) lane 2) with little effect on HEK 293 cells (FIG. 16 (B) lane 2). In contrast, the addition of DBM significantly increased the levels of HIF-1α in both cell lines (FIGS. 16 (A) and (B) lane 3). The simultaneous exposure to DBM plus Zn²⁺ completely abrogated hypoxia-induced elevations in HIF-1α levels (FIGS. 16 (A) and (B) lane 4). The addition of ferric iron (Fe³⁺, another metal that binds to DBM with high affinity [21]) did not significantly alter the effect of DBM on HIF-1α levels in HT 144 cells (FIG. 16 (A) lane 5), but decreased slower migrating isoforms of HIF-1α in HEK 293 cells (FIG. 16 (B) lane 5).

The mRNA of HIF-1α is produced continuously and is not usually affected by oxygen or stress as compared to the HIF-1α protein subunit that is rapidly degraded in normoxic cells. However, one form of HIF-1α mRNA has been designated HIF-1αZ because it exhibits a loss of exon 12 in response to Zn²⁺ ions at concentrations above 100 μM, and functions in a dominant negative manner [22]. Conceivably, induction of HIF-1αZ could occur at lower concentrations of Zn²⁺ in the presence of DBM.

The effects of DBM alone, Zn²⁺ alone and DBM plus Zn²⁺ on HIF-1α mRNA levels were assessed by semiquantitative PCR in extracts of HEK 293 kidney cells under normoxia for 2 hours in serum free media (FIG. 17). HIF-1α mRNA levels were not significantly different from baseline (FIG. 17, lane 1) when cells were exposed to DBM alone (FIG. 17, lane 2) or Zn²⁺ alone (FIG. 17, lane 3). Treatment with DBM plus Zn²⁺ (FIG. 17, lane 4) resulted in a small decrease in the PCR product, though possibly not to a degree sufficient to account for the almost total absence of HIF-1α observed on Western blots. HIF-1αZ mRNA levels were not found to be increased (data not shown).

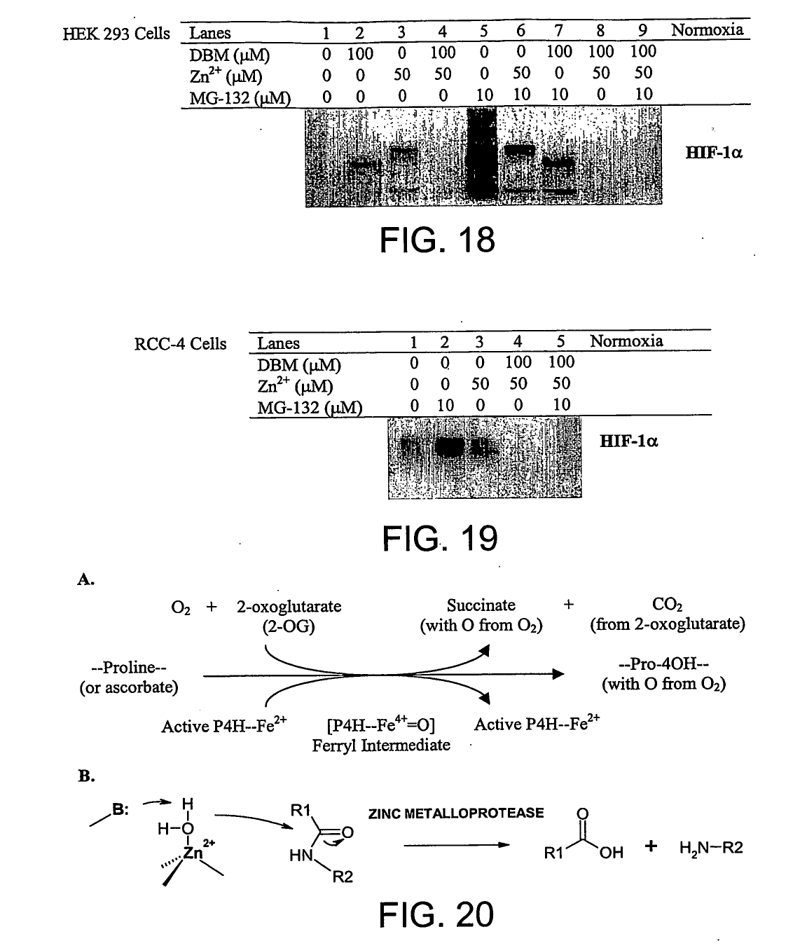

Proteosomal degradation of HIF-1α was assessed in HEK 293 embryonic kidney cells treated with DBM alone, Zn²⁺ alone or the combination under ambient oxygen in the presence or absence of the proteosome inhibitor MG-132 (FIG. 18). Though HIF-1α was essentially undetectable in cells exposed to ambient oxygen alone for 2 hours (FIG. 18, lane 1), it was readily detected when either DBM (FIG. 18, lane 2) or Zn²⁺ (FIG. 18, lane 3) was added. Of note, Zn²⁺ induced the stabilization of HIF-1α isoforms that are of higher molecular weight than those induced by DBM. The combination of DBM and Zn²⁺ resulted in the complete reversal of HIF-1α protein stabilization observed when these agents were used individually (FIG. 18, lanes 4 and 8). Proteosomal inhibition with MG-132 resulted in the accumulation of a broad range of proteins that possibly represent ubiquitinated forms of HIF-1α [20] (FIG. 18, lane 5). However, the addition of MG-132 to either Zn²⁺ (FIG. 18, lane 6) or DBM (FIG. 18, lane 7) did not stabilize these other forms of HIF-1α. Furthermore, MG-132 did not prevent the reversal of induced HIF-1α stabilization observed when DBM and Zn²⁺ are combined (FIG. 18, lane 9). The HIF-1α isoforms are more clearly resolved on the gel from FIG. 18 than FIG. 15. This may be due to differences in the time the gels were run.

The potential role of the VHL gene product in mediating the effects of DBM and Zn²⁺ on HIF-1α protein levels was explored in RCC-4 renal cell carcinoma cells that are VHL (−/−) and do not express the VHL protein. As expected, HIF-1α was detected by Western blot under normoxic conditions (FIG. 19, lane 1). Stabilization of HIF-1α was enhanced by exposure to MG-132 for 1 hour (FIG. 19, lane 2) and, to a lesser degree, by exposure to Zn²⁺ (FIG. 19, lane 3). Simultaneous treatment with DBM and Zn²⁺ resulted in loss of HIF-1α detection (FIG. 19, lane 4) that was not reversed by MG-132 (FIG. 19, lane 5). These experiments have not been performed as yet in RCC4 cells with DBM alone or in RCC4 cells transfected with pVHL.

Discussion

Basal levels of HIF-1α are low in cells exposed to ambient oxygen, yet under these conditions both DBM and Zn²⁺ stabilize HIF-1α and cause the accumulation of HIF-1α protein (FIG. 15). It has been shown that this stabilizing effect on HIF-1α was itself reversed by the concomitant addition of Zn²⁺ to DBM under ambient oxygen. Simultaneous exposure of cells to DBM plus Zn²⁺ resulted in rapid decreases in detectable levels of HIF-1α (<1 hr). Since this reversal in HIF-1α stabilization was observed when either DBM or Zn²⁺ was present in excess, one may conclude that DBM and Zn²⁺ did not simply counteract each other through chelation. This destabilizing effect appears unique to the combination of DBM plus Zn²⁺ since it was not observed with other metals known to form chelates with DBM such as Fe³⁺, Co²⁺, Cd²⁺ and Ni²⁺. The fact that HIF-1α Z mRNA was not detected suggests that the destabilization of HIF-1α was not mediated by this splice variant. Though assessment of downstream effectors of HIF-1α (e.g., VEGF) was not included in the preliminary data, the effects of DBM and cobalt on VEGF in LNCaP human prostate cancer cells have been previously reported [20].

Though Zn²⁺ appears to stabilize HIF-1α in normoxic cells (FIGS. 15 and 18), it may counteract the effect of hypoxia-induced stabilization of HIF-1α (FIG. 16, lane 2). This suggests that there may be differences in the way DBM and Zn²⁺ interact with HIF-1α under normoxic and hypoxic conditions.

DBM plus Zn²⁺ induced the loss of HIF-1α in RCC-4 cells that lack pVHL (FIG. 19, lane 4). This supports the conclusion that the classical VHL-dependent proteosomal degradation pathway (FIG. 15 (A)) is not involved in this process. VHL-independent pathways for the ubquitination of HIF-1α in protein degradation have been described [23]. The Hsp90 pathway inhibited by geldanamycin showed a decrease in HIF-1α in renal cell carcinoma cell lines lacking VHL [19]. In this setting, the loss of HIF-1α typically occurred after 8 hrs of incubation, but still involved ubiquitination and proteosomal degradation. Accumulation of p53 was observed under conditions of severe and prolonged (16 hr) hypoxia [24, 25].

VHL-independent ubiquitination of HIF-1α has been reported [26]. This would allow VHL-independent proteosomal degradation of HIF-1α to occur. Also, HIF-1α could undergo degradation by the p53, Mdm2, E3 ligase pathway of proteosomal destruction in a VHL-independent manner. This could provide an alternate pathway for the degradation of HIF-1α by the 26S proteosome. However, it was found that HIF-1α is lost in RCC-4 cells treated with DBM plus Zn²⁺ plus MG-132 (FIG. 19, lane 5). Therefore, degradation of HIF-1α in this context appears to occur by a mechanism that is both VHL-independent and proteosome-independent.

Transcriptional down-regulation has been described for HIF-1α and thus represents a possible VHL-independent, proteosome-independent mechanism for regulating HIF-1α levels [27]. However, the preliminary data does not support this mechanism since HIF-1α mRNA levels did not decrease in cells treated with DBM plus Zn²⁺ to a degree sufficient to explain our findings of undetectable levels of HIF-1α protein (FIG. 17). Nevertheless, this mechanism cannot be discounted without closer scrutiny.

Translational control has not been described for HIF-1α and represents an unlikely explanation for the rapid loss of HIF-1α in cells treated with DBM plus Zn²⁺. However, this type of mechanism cannot be excluded since the data does not rule out activation of unspecified proteolytic pathways by DBM plus Zn²⁺.

Proteosome-independent down-regulation may involve decreased synthesis of HIF-1α or degradation by alternate pathways. One of the major alternate pathways for proteolysis involves the Zn²⁺ metalloproteases. The prolyl-4-hydroxylation reaction catalyzed by HIF P4H (FIG. 20 (A)) is compared to proteolytic reactions catalyzed by Zn²⁺ metalloproteases (FIG. 20 (13)).

HIF P4H is a dioxygenase that requires Fe²⁺, 2-oxoglutarate (2-OG), O₂ and a proline-containing peptide to catalyze prolyl-4-hydroxylation (FIG. 20 (A)). The reaction products include succinate and CO₂. One of the atoms from ambient O₂ is incorporated into succinate and the other into Pro-4OH. A proline-containing peptide is not absolutely required as a substrate for this reaction. HIF P4H can convert ascorbate to dehydroascorbic acid if no proline-containing substrate is present [28]. The prolyl-4-hydroxylation reaction has been studied in detail in the collagen P4H. Superoxo, peroxo and ferryl (Fe⁴⁺═O) catalytic intermediates generate a highly reactive hydroxyl radical that is precisely directed to replace the trans proton on C-4 of specific proline residues [29].

Zn²⁺ plays a crucial role in various biochemical systems. Many proteins can only assume an active configuration when Zn²⁺ is bound to specific structural sites (e.g., zinc finger regions). Zn²⁺ also serves as the active metal in catalytic sites as well as co-catalytic sites [30]. In Zn²⁺ co-catalytic sites, a Zn²⁺ atom forms one or more bridges to a second Zn²⁺ atom or to a different metal by bonding to a common amino acid (usually histidine or aspartate) or to H₂O. Conceivably, DBM plus Zn²⁺ could make HIF-1α susceptible to down-regulation by inducing RNA inhibitors. Alternatively, DBM plus Zn²⁺ may stimulate non-proteosomal proteases to degrade HIF-1α. In addition, it is possible that DBM plus Zn²⁺ directly participates in the degradation of HIF-1α.

When functioning as a catalytic metal in biological systems, Zn²⁺ exclusively catalyzes the deprotonation of water to form Zn²⁺-bound hydroxide ions (FIG. 20 (B)), however, this step is only one component of many diverse catalytic reactions. For example, carbonic anhydrase uses Zn²⁺ to generate hydroxide ions (OH⁻) from water for nucleophilic attack on CO₂ to form bicarbonate (HCO₃⁻). Other enzymes such as carboxypeptidase A (a digestive enzyme), angiotensin-converting enzyme (ACE) and the matrix metalloproteases (MMPs), use Zn²⁺ and water in a similar manner to degrade a wide range of proteins. DBM plus Zn²⁺ may stimulate Zn²⁺ metalloproteases to cleave HIF-1α, though carboxypeptidase A and ACE have relatively restricted access to their active sites. The MMPs, a family of at least 26 endopeptidases that cleave a variety of proteins, are more likely candidates in this regard [30].

Direct participation of DBM and Zn²⁺ in the degradation of HIF-1α represents a remote, but intriguing possibility. In this scenario, DBM and Zn²⁺ are incorporated into the active site of HIF P4H. The Fe²⁺ catalytic site is transformed into a Fe²⁺—Zn²⁺ co-catalytic site. Catalytic activity shifts from Fe²⁺ to Zn²⁺ allowing Zn²⁺-mediated endopeptidase activity to take place with specificity for HIF-1α bound to its native binding site.

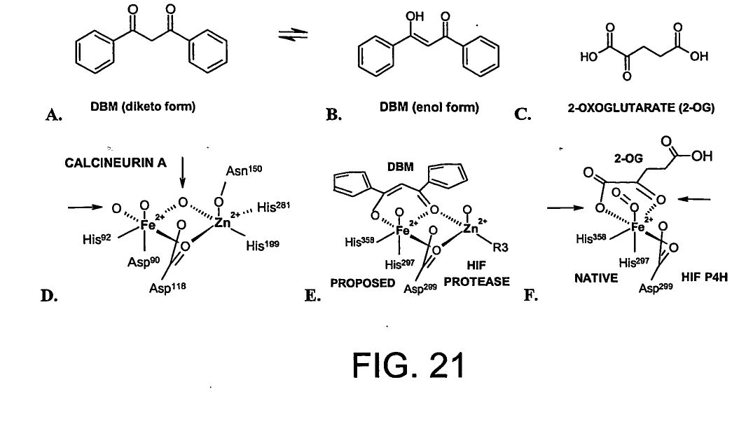

The proposed co-catalytic site for this novel “HIF-1α Protease” is depicted in FIG. 21 (E). When used independently, DBM (FIGS. 21 (A) and (B)) and Zn²⁺ inhibit HIF P4H (FIG. 21 (F)) and stabilize HIF-1α. In this model, the co-catalytic site can only form when DBM and Zn²⁺ are present at the same time (FIG. 21 (E)) making simultaneous binding a prerequisite for proteolytic activity. In native HIF P4H (FIG. 21 (F)), endogenous ligands (His²⁹⁷, Asp²⁹⁹ and His³⁵⁸) occupy 3 of 6 coordination sites on Fe²⁺ and hold it in the active site [28]. The two arrows indicate the location of 2-OG subsite II, the site where 2-OG (FIG. 21 (C)) binds to Fe²⁺ [29, 31]. The sixth coordination site is occupied by O₂. As depicted, DBM binds to Fe²⁺ at 2-OG subsite II via oxygen atoms on C-1 and C-3 of DBM (FIG. 21 (E)). One of the oxygen atoms from DBM and one from Asp²⁹⁹ bind Zn²⁺ to Fe²⁺ by forming bridges that firmly anchor Zn²⁺ to a four-member ring in the newly formed Fe²⁺—Zn²⁺ co-catalytic site. This configuration of ligand binding is similar to the pattern found in the co-catalytic site of calcineurin A (FIG. 21 (D)), a naturally occurring metallophosphatase [32]. In calcineurin A, Fe²⁺ rather than Zn²⁺ is the catalytic metal.

The proposed Zn²⁺-mediated co-catalytic activity begins with H₂O or O₂ in the sixth coordination site on Fe²⁺ (FIG. 21 (E), protons omitted for clarity). Another H₂O molecule is bound to Zn²⁺. R₃ represents H₂O or possibly a carboxyl or amino group from another ligand that is also coordinated to Zn²⁺. This starting configuration is stabilized by hydrogen bounding between H₂O or O₂ on Fe²⁺, H₂O on Zn²⁺ and a proton or electron pair on the R₃ ligand. Endopeptidase activity is generated when peptide carbonyl groups in HIF-1α undergo nucleophilic attack by Zn²⁺-bound hydroxide ions as described in FIG. 20 (B). Thermodynamically, this type of proteolysis is preferred to the native reaction that requires oxidation of Fe²⁺ to Fe⁴⁺ and hydroxylation of an aliphatic carbon in proline (FIG. 20 (A)). Since H₂O is the substrate for this form of proteolysis, the reaction can occur in the presence or absence of O₂. This model is a working hypothesis that is consistent with all experimental data that have been obtained to date. It is plausible as are the other possible mechanisms previously described.

The HIF P4Hs form a subfamily of three isoenzymes in mammalian cells known as prolyl hydroxylase domain (PHD) proteins that have been designated PHD1, PHD2 and PHD3 [33]. PHDs are non-equilibrium enzymes in the sense that they do not catalyze the reverse reaction. Berra and colleagues have shown that all three isoforms are expressed to varying degrees in a wide range of immortalized and non-immortalized human cells of different origin and that PHD2 is the critical oxygen sensor that maintains the low steady-state levels of HIF-1α in normoxia [34]. This battery of cell lines included CAL27 (squamous cell carcinoma of the tongue), CAL51 (breast cancer), HaCAT (keratinocyte), HT29 (colon cancer), RCC4/pVHL (clear cell renal cell carcinoma with reintroduced wild type pVHL), WM9 (melanoma), FHN (fibroblasts), umbilical vein (HUVEC) and HeLa (uterine cancer) cells.

The cellular expression patterns of PHD isoforms have recently been reported for cell lines dervived from additional human tissue types including BxPC-3 (pancreatic carcinoma), PC-3 (prostate carcinoma), MCF7 (breast cancer), HS-587T (breast cancer), MDA-435 (breast cancer), T47D (breast cancer), ZR-75-1 (breast cancer), U-2 OS (osteosarcoma), OVCAR-3 (ovarian carcinoma), A549 (lung carcinoma), HT1080 (rhabdomyosarcoma), JAR (choriocarcinoma), BT-474 (breast cancer), 833K (testicular cancer) and SuSa (testicular cancer) cell lines [35]. These investigators observed major variations in expression patterns, even within cell lines derived from a single type of tissue (breast cancer) with PHD3 frequently below the limits of detection. Overall, PHD1 and PHD3 mRNA and protein levels were relatively low across a wide range of normoxic cells thus confirming initial reports that PHD2 was the most abundant isoenzyme in normoxic culture, though the induction of PHD3 was found to be more striking under hypoxia. Small inhibitory RNAs (siRNAs) were used in this study to demonstrate that each PHD isoform plays a role that is not redundant with the others under a given set of culture conditions with the relative contribution of each strongly dependent on its abundance [35].

Specific siRNAs have been synthesized that are capable of individually silencing each PHD isoform without affecting the expression of the other isoforms [34-36]. Silencing PHD2 alone is sufficient to stabilize and activate HIF-1α [34]. If DBM and Zn²⁺ must bind to HIF P4H (PHD isoforms 1-3) as depicted in FIG. 21 (E) for the paradoxical down-regulation of HIF-1α to occur, siRNAs to PHD1, PHD2 and/or PHD3 should reverse the effect of DBM plus Zn²⁺.

### REFERENCES

[1] J. I. Bardos, M Ashcroft, Hypoxia-inducible factor-1 and oncogenic signaling, BioEssay 26 (2004) 262-269.
[2] G. Semenza, Targeting HIF-1 for Cancer Therapy, Nat Rev Cancer 3 (2003) 721-732.
[3] M. Ivan, T. Haberberger, D. C. Gervasi, K. S. Michelson, V. Gunzler, K. Kondo, H. Yang, I. Sorokino, R. C. Conaway, J. W. Conaway, W. G. Kaelin, Biochemical purification and pharmacological inhibition of a mammalian prolyl hydrolyase acting on hypoxia-inducible factor, Proc Natl Acad Sci 99(2002) 13459-13464.
[4] M. Ivan, K. Kondo, H. Yang, W. Kim, J. Valiando, M. Ohhl, A. Salic, M. Asara, W. S. Lane, W. G. Kaelin; HIF-1α targeted for VHL-mediated destruction by proline hydroxylation: implications for O2 sensing, Science 292(2001) 464-468.
[5] P. Jaakola, D. R. Mole, Y. M. Tian, M. I. Wilson, J. Gielbert, S. J. Gaskell, A. Kriegsheim, H. F. Hebestreit, M. Mukherji, C. J. Schofield, P. H. Maxwell, C. W. Pugh, P. J. Ratcliffe, Targeting of HIF-1α to the von Hippel-Lindau ubiquitylation complex by O₂-regulated prolyl hydroxylation, Science 292(2001) 468-472.
[6] F. Yu, S. B. White, Q. Zhao, F. S. Lee, HIF-1α binding to VHL is regulated by stimulus-sensitive proline hydroxylation, Proc Natl Acad Sci 98 (2001) 9630-9635.
[7] J. W. Jeong, M. K. Bae, M. Y. Ahn, S. H. Kim, T. K. Sohn, M. H. Bae, M. A. Yoo, E. J. Song, K. W. Kim, Regulation and destabilization of HIF-1α by ARD-1 mediated acetylation, Cell 111 (2002) 709-20.
[8] L. A. McNeill, K. S. Hewitson, T. D. Claridge, J. F. Seibel, L. E. Horsfall, C. J. Schofield, Hypoxia-inducible factor asparaginyl hydroxylase (FIH-1) catalyses hydroxylation at the beta-carbon of asparagine-803, Biochem J. 367 (2002) 571-5.
[9] J. C. Yang, L. Haworth, R. M. Sherry, P. Hwu, D. J. Schwartzentruber, S. L. Topalian, S. M. Steinberg, H. X. Chen, and Steven A. Rosenberg. A Randomized Trial Of Bevacizumab, An Anti-Vascular Endothelial Growth Factor Antibody, For Metastatic Renal Cancer. N Engl J Med 349 (2003) 427-434.
[10] H. Zhong, A. M. De Marzo, E. Laughner, M. Lim, D. A. Hilton, D. Zagzag, P. Buechler, W. B. Issacs, G. L. Semenza, J. W. Simons, Overexpression of hypoxia inducible factor 1alpha in common human cancers and their metastases, Cancer Res 59(1999) 5830-5.
[11] A. Rapisarda, B. Uranchimeg, D. A. Scudiero, M. Selby, E. A. Sausville, R. H. Shoemaker, G. Melillo, Identification of small molecule inhibitors of hypoxia-inducible factor 1 transcriptional activation pathway, Cancer Res. 62(2002) 4316-24.
[12] A. Rapisarda, B. Uranchimeg, O. Sordet, Y. Pommier, R. H. Shoemaker, G. Melillo, Topoisomerase I-mediated inhibition of hypoxia-inducible factor 1: mechanism and therapeutic implications, Cancer Res. 64(2004) 1475-82.
[13] N. J. Mabjeesh, D. Esculin, T. M. LaVallee, V. S. Pribluda, G. M. Swartz, M. S. Johnson, M. T. Willard, H. Zhong, J. W. Simons, P. Giannakakou, 2ME2 inhibits tumor growth and angiogenesis by disrupting microtubules and dysregulating HIF, Cancer Cell 3 (2003) 363-374.
[14] S. J. Welsh, R. R. Williams, A. Birmingham, D. J. Newman, D. L. Kirkpatrick, G. Powis, The Thioredoxin Redox Inhibitors 1-Methylpropyl 2-inidazolyl Disulfide and Pleurotin Inhibit Hypoxia-induced Factor 1α and Vascular Endothelial Growth Factor Formation, Mol Cancer Therap 2(2003) 235-243.
[15] E. J. Yeo, Y. S. Chun, Y. S. Cho, J. Kim, J. C. Lee, M. S. Kim, J. W. Park, YC-1: a potential anticancer drug targeting hypoxia-inducible factor 1, Natl Cancer Inst. 95(2003) 516-2.
[16] A. L. Kung, S. Wang, J. M. Klco, W. G. Kaelin, D. M. Livingston, Suppression of tumor growth through disruption of hypoxia-inducible transcription, Nat Med 6 (2000) 1335-40.
[17] N. J. Mabjeesh, D. E. Post, M. T. Willard, B. Kaur, E. G. Van Meir, J. W. Simons, H. Zhong, Gledanamycin induces degradation of hypoxia-inducible factor 1 alpha protein via the proteasome pathway in prostate cancer cells, Cancer Res 62(2002) 2478-82.
[18] J. S. Isaacs, Y. J. Jung, E. G. Mimnaugh, A. Martinez, F. Cuttitta, L. M. Neckers, Hsp90 regulates a von Hippel Lindau-independent hypoxia-inducible factor-1 alpha degradative pathway, J Biol Chem. 277 (2002) 29936-44.
[19] N. J. Mabjeesh, M. T. Willard, W. B. Harris, H. Y. Sun, R. Wang, H. Zhong, J. N. Umbreit, J. W. Simons, Dibenzoylmethane, a natural dietary compound, induces HIF-1 alpha and increases expression of VEGF, Biochem Biophys Res Commun, 303 (2003) 279-86.
[20] Yuki H, Hirano N, Kawasaki H, Yajima T, Analysis of serum iron by gel permeation high-performance liquid chromatography, J Chrom 221 (1980) 271-7.
[21] Y. S. Chun, E. J. Choi, E. J. Yeo, J. H. Lee, M. S. Kim, J. W. Park, A new HIF-1 alpha variant induced by Zn²⁺ suppresses HIF-1 mediated hypoxic responses, J Cell Sci 114(2001) 4051-61.
[22] P. H. Maxwell, M. S. Wiesener, G. W. Chang, S. C. Clifford, E. C. Vaux, M. E. Cockman, C. C. Wykoff, C. W. Pugh, E. R. Maher, P. J. Ratcliffe, The tumor suppressor protein VHL targets hypoxia-inducible factors for oxygen-dependent proteolysis, Nature 399 (1999) 203-4.
[23] J. S. Isaacs, Y. J. Jung, E. G. Mimnaugh, A. Martinez, F. Cuttitta, L. M. Neckers, Hsp90 regulates a von Hippel Lindau-independent hypoxia-inducible factor-1 alphadegradative pathway, J. Biol Chem. 277(2002) 29936-44.
[24] D. Chen, M. Li, J. Luo, W. Gu, Direct interactions between HIF-1α and Mdm2 modulate p53 function, J. Biol. Chem. 278 (2003) 13595-13598.
[25] T. Schmid, J. Zhou, R. Kohl, B. Brune, p300 relieves p53-evoked transcriptional repression of hypoxia-inducible factor-1 (HIF-1), Biochem J. 380 (2004) 289-95.
[26] K. Kanaya, T. Kamitani, pVHL-independent ubiquitination of HIF1alpha and its stabilization by cobalt ion, Biochem Biophys Res Commun. 306(2003) 750-5.
[27] T. T. Tang, L. A. Lasky, The forkhead transcription factor FOXO4 induces the down-regulation of hypoxia-inducible factor 1 alpha by a von Hippel-Lindau protein independent mechanism, J Biol Chem. 278(2003) 30125-35.
[28] Hirsilä M, Koivunen P, Günzler V, Kivirikko K I and Myllyharju J. Characterization of the human prolyl 4-hydroxylases that modify the hypoxia-inducible factor. J Biol Chem 278:30772-30780 (2003).
[29] Hanauske-Abel H M and Günzler V. A stereochemical concept for the catalytic mechanism of prolyhydroxylase: Applicability to classification and design of inhibitors. J Theor Biol 94:421-455 (1982).
[30] Auld D S. Zinc coordination sphere in biochemical zinc sites. BioMetals 14:271-313 (2001).
[31] Myllyharju J and Kivirikko K I. Characterization of the iron- and 2-oxoglutarate-binding sites of human prolyl 4-hydroxylase. EMBO J 16:1173-1180 (1997).
[32] Mertz P, Yu L, Sikkink R and Rusnak F. Kinetic and spectroscopic analyses of mutants of a conserved histidine in the metallophosphatases calcineurin and λ protein phosphatase. J Biol Chem 272:21296-21302 (1997).
[33] Soldav D V, Henegouwen A T, Enright G D, Ratcliffe C I and Ripmeester J A. Nickel(II) and zinc(II) dibenzoylmethanates: Molecular and crystal structure, polymorphism, and guest- or temperature-induced oligomerization. Inorg Chem 40:1626-1636 (2001).

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

Therefore, the following is claimed:

1. A method of treating cancer or a tumor comprising administering to a host in need of treatment an effective amount of at least one HIF-1 inhibitor composition, wherein the HIF-1 inhibitor composition comprises a bidentate zinc chelate.

2. The method of claim 1, wherein the bidentate zinc chelate comprises a beta-diketone bidentate zinc chelate compound.

3. The method of claim 2, wherein the beta-diketone bidentate zinc chelate compound has the following structure: 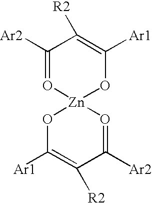
wherein Ar₁ and Ar₂ each are individually selected from at least one of the following: 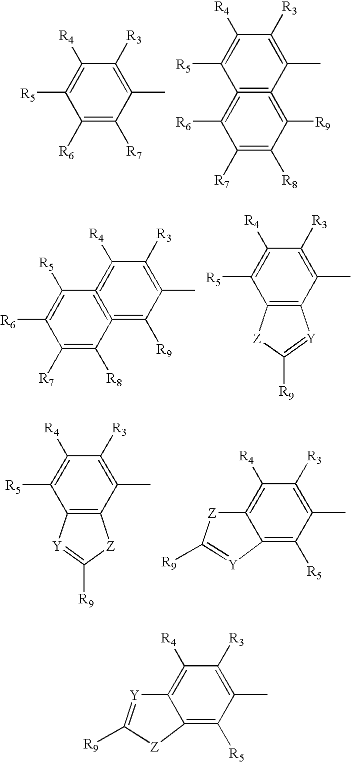
wherein R2, R3, R4, R5, R6, R7, R8, and R9 are each individually selected from at least one of: hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

4. The method of claim 3, wherein the beta-diketone compound comprises a dibenzoylmethane-type compound.

5. The method of claim 3, wherein the dibenzoylmethane-type compound comprises dibenzoylmethane.

6. The method of claim 1, wherein the bidentate zinc chelate has the following structure: 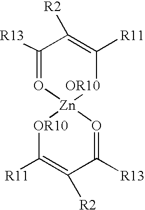
wherein R10 is selected from hydrogen and a sulfonyl group, and wherein R2, R11, and R13 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

7. The method of claim 1, wherein the bidentate zinc chelate has the following structure: 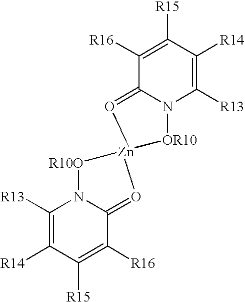
wherein R10 is selected from hydrogen and a sulfonyl group, and wherein R13, R14, R15, and R16 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

8. The method of claim 1, wherein the bidentate zinc chelate has the following structure: 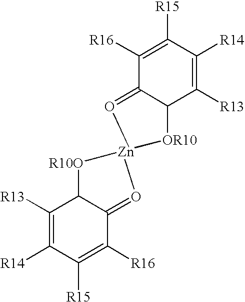
wherein R10 is selected from hydrogen and a sulfonyl group, and wherein R13, R14, R15, and R16 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

9. The method of claim 1, wherein the bidentate zinc chelate has the following structure: 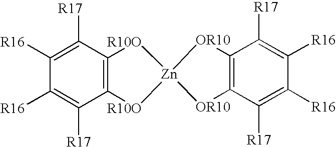
wherein R10 is selected from hydrogen and a sulfonyl group, and wherein R16 and R17 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

10. The method of claim 1, wherein the bidentate zinc chelate has the following structure: 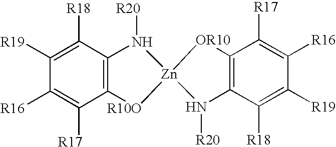
wherein R10 and R20 are selected from hydrogen and a sulfonyl group, and wherein R16, R17, R18, and R19 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

11. The method of claim 1, wherein the bidentate zinc chelate has the following structure: 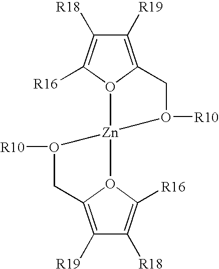
wherein R10 is selected from hydrogen and a sulfonyl group, and wherein R16, R18, and R19 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

12. The method of claim 1, wherein the bidentate zinc chelate has the following structure: 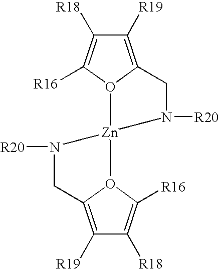
wherein R20 is selected from hydrogen and a sulfonyl group, and wherein R16, R18, and R19 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

13. The method of claim 1, wherein the bidentate zinc chelate has the following structure: 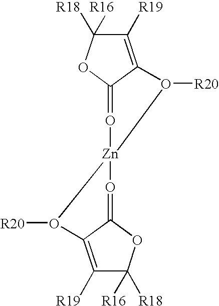
wherein R10 is selected from hydrogen and a sulfonyl group, and wherein R16, R18, and R19 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

14. The method of claim 1, wherein the bidentate zinc chelate has the following structure: 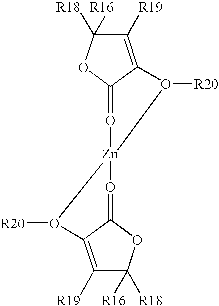
wherein R20 is selected from hydrogen and a sulfonyl group, and wherein R16, R18, and R19 are each individually selected from hydrogen, alkyl groups, aryl groups, halo groups, hydroxy groups, alkoxy groups, alkylamino groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons.

15. The method of claim 1, 3, and 6 through 13, wherein the cancer or tumor is selected from the group consisting of bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, and small-cell lung cancer.

16. The method of claim 1, 3, and 6 through 13, further comprising treating the host with at least one conventional anticancer treatment chosen from radiation and chemotherapy.

17. The method of claim 1, 3, and 6 through 13, further comprising treating the host with at least one conventional anticancer agent selected from an antibiotic, anti-inflammatory, anti-oxidant, analgesic, radioisotope, nascopine, paclitaxel, nocodazole, vinca alkaloids, adriamycin, alkeran, Ara-C, BiCNU, busulfan, CCNU, carboplatinum, cisplatinum, cytoxan, daunorubicin, DTIC, 5-FU, fludarabine, hydrea, idarubicin, ifosfamide, methotrexate, mithramycin, mitomycin, mitoxantrone, nitrogen, mustard, velban, vincristine, VP-16, gemcitabine, herceptin, irinotecan, camptosar, CPT-11, leustatin, navelbine, rituxan, STI-571, taxotere, topotecan, hycamtin, xeloda capecitabine, zevelin, and combinations thereof.

18. The method of claim 2, wherein the beta-diketone compound includes pharmaceutically acceptable salts of the beta-diketone compounds, pharmaceutically acceptable prodrugs of the beta-diketone compounds, beta-diketone compound derivatives, and combinations thereof.

19. The method of claim 18, wherein the beta-diketone compound includes a dibenzoylmethane-type compound, and wherein the dibenzoylmethane-type compound includes pharmaceutically acceptable salts of the dibenzoylmethane-type compounds, pharmaceutically acceptable prodrugs of the dibenzoylmethane-type compounds, dibenzoylmethane-type compound derivatives, and combinations thereof.

20. A chemopreventative method of prophylactically treating cancers or tumors comprising administering to a host in need of treatment an effective amount of at least one bidentate zinc chelate of claims 3 and 6 through 14.

21. The chemopreventative method of claim 20, wherein the bidentate zinc chelate includes pharmaceutically acceptable salts of the bidentate zinc chelate, pharmaceutically acceptable prodrugs of the bidentate zinc chelate, bidentate zinc chelate derivatives, and combinations thereof.

22. The chemopreventative method of claim 20, wherein the bidentate zinc chelate includes a hydrolysis, an oxidation, or a reduction reaction product of the bidentate zinc chelate.

23. A pharmaceutical composition comprising at least one bidentate zinc chelate in combination with a pharmaceutically acceptable carrier, wherein the at least one bidentate zinc chelate is present in a dosage level effective to treat cancers or tumors of claims 3 and 6 through 14.

24. The pharmaceutical composition of claim 23, wherein the bidentate zinc chelate includes pharmaceutically acceptable salts of the bidentate zinc chelate, pharmaceutically acceptable prodrugs of the bidentate zinc chelate, bidentate zinc chelate derivatives, and combinations thereof.

25. The pharmaceutical composition of claim 23, wherein pharmaceutical composition can be administered orally, rectally, parenterally, intrasystemically, intravaginally, intraperitoneally, topically, and bucally.

26. The pharmaceutical composition of claim 23, wherein the bidentate zinc chelate includes a hydrolysis, a oxidation, or a reduction reaction product of the bidentate zinc chelate.

27. A method for the treatment or prevention of a hypoxia-related pathology comprising: administering to a host in need of such treatment an HIF-1 inhibiting amount of at least one of the bidentate zinc chelate of claims 3 and 6 through 14.

28. A method of modulating HIF-1 activity in a cell comprising: contacting the cell with an HIF-1 inhibiting amount of at least one of the bidentate zinc chelate of claims 3 and 6 through 14.

29. A method of downregulating HIF-1 activity in a cell comprising: contacting the cell with an HIF-1 inhibiting amount of at least one of the bidentate zinc chelate of claims 3 and 6 through 14.

30. A method of treating or preventing cancer or a tumor in a host comprising administering to the host a HIF-1 inhibiting amount of at least one of the bidentate zinc chelate of claims 3 and 6 through 14.

31. A method of modulating gene transcription in a cell comprising contacting the cell with an HIF-1 inhibiting amount of at least one of the bidentate zinc chelate of claims 3 and 6 through 14.

32. The method of claim 31, wherein the cell is a cancer cell.

33. The method of claim 31, wherein the cell is a tumor cell.
